(19) United States
(12) Patent Application Publication
(10) Pub. No.: US 2026/0317249 A1
(43) Pub. Date: Oct. 15, 2026

(54) AGRICULTURAL COMPOSITION AND METHOD OF USE THEREOF IN UPREGULATING GENE EXPRESSION

(71) Applicant: UPL Mauritius Limited, Port Louis (MU)

(72) Inventor: Michael Lloyd Jones, Research Triangle Park, NC (US)

(21) Appl. No.: 19/648,750

(22) Filed: Apr. 15, 2026

Related U.S. Application Data
(60) Provisional application No. 63/789,071, filed on Apr. 15, 2025.

(51) Int. Cl. C05F 11/00; C05B 7/00; C05C 9/00

(52) U.S. Cl. CPC C05F 11/00; C05B 7/00; C05C 9/00

(57) ABSTRACT

This application relates to the field of agricultural compositions. This application discloses a composition comprising at least one plant extract, at least one weak acid, an oligosaccharide and an amino acid or an amino acid source for increasing nutrient use efficiency and abiotic stress tolerance. This application further discloses method for regulating expression of genes and pathways associated with nutrient use efficiency, cellular/subcellular uptake, long-distance transport of essential resources and abiotic stress tolerance comprising applying the agrochemical composition. The present application discloses agricultural treatments and methods that induce component-driven, temporally staged, and tissue-specific molecular reprogramming in plants, resulting in enhanced nutrient use efficiency, improved nitrogen status and nitrogen content, coordinated transporter and hormonal regulation, increased stress resilience without sustained growth penalty, and improved plant growth and performance.

### CROSS-REFERENCE TO RELATED APPLICATION

This application claims the benefit of and priority to U.S. Provisional Application No. 63/789,071, filed Apr. 15, 2025, the entire contents of which are incorporated by reference herein.

### FIELD OF THE INVENTION

This application relates in general to the field of agricultural compositions comprising at least one plant extract, at least one weak acid, an amino acid or a source of amino acid, an oligosaccharide and one or more adjuvants. In particular, the instant application also relates to upregulation of expression of at least one gene selected from the group comprising aquaporins, heat shock proteins, WRKY transcription factors, auxins, trehalose, and peroxidases. Further, this application discloses methods for increasing nutrient efficiency, translocation and uptake of nutrients, and abiotic stress tolerance and a combination thereof.

### BACKGROUND

Agrochemical compositions comprising plant growth promoters such as nitrogen, phosphorus and/or potassium when used as fertilizers offer benefits in nutrition management through their strong potential to increase nutrient use efficiency. Nutrients in such compositions are released very slowly as compared to conventional fertilizers both in soil when applied as broadcast or in solution when applied as foliar spray. This approach not only increases nutrient-use efficiency but also minimizes nutrient leaching into ground water.

Yield is an aggregated trait built, not only, on the genetics of the seed and cultivated plants, but also, and perhaps more importantly, the interactions of that cultivated plants with the farmer's inputs and the natural environment in which the crop is grown. With an ever-growing human population and an estimated 800 million food insecure people in the world today, minimizing the impact of these interactions on yield potential is critical.

In the 20th century, the use of synthetic fertilizers was adopted globally and while these inputs are expensive to the farmer the yield boost from synthetic fertilizers resulted in high application rates of both nitrogen and phosphorous. These high application rates have increased yield substantially; however, the environmental cost of low nutrient use efficiency has had big impacts on water quality around the world. The United Nations (UN) Environment Program has identified excessive reactive N as one of five emerging threats facing the planet (Sutton et al., 2019), such that the fourth UN Environment Assembly (March 2019) adopted a resolution on “Sustainable N management” and published a road map for responsible use of agricultural nitrogen that highlighted, among other things, the need for enhanced-efficiency fertilizers.

In this regard, increasing the efficiency of any given nutrients to the plants are crucial to reach to the Global guidance and goals of UN, FAO and other international organizations in terms of securing food security in a sustainable and economically viable manner. Thus, there is a need in the art for new proprietary nutrient delivery technology to reach some global guidance and goals to ensure global food security in a sustainable and economically viable way.

Therefore, it is an object of the present application to provide an agrochemical composition that regulates expression of genes and pathways associated with abiotic stress tolerance, nutrient use efficiency and water use efficiency. The inventors of the instant application found that the composition disclosed herein achieves the needed benefit of increase in yield potential (via increase in abiotic stress tolerance, nutrient use efficiency and water use efficiency) and/or yield.

### SUMMARY

An embodiment disclosed herein provides an agrochemical composition comprising:
a) at least one plant extract;
b) at least one weak acid;
c) an oligosaccharide;
d) an amino acid or an amino acid source; and
e) one or more adjuvant.

In another embodiment disclosed herein, provided is an agrochemical composition comprising:
a) at least one plant extract;
b) at least one weak acid;
c) an oligosaccharide;
d) an amino acid or an amino acid source; and
e) one or more adjuvant;
f) optionally, a nutrient particle or a plurality of nutrient particles, wherein the nutrient particle is a plant nutrient, selected from nitrogen, its compounds or salts; phosphorus, its compounds or salts; potassium, its compounds or salts; or combinations thereof, and
g) optionally, a humic substance selected from humic acid, fulvic acid, or combinations thereof.

In another embodiment, an agrochemical composition disclosed herein upregulates expression of at least one gene associated with a biochemical or a physiological pathway selected from the group consisting of Amino Acid Transport and Metabolism, Anti-Oxidant Activity, Aquaporin Biosynthesis, Auxin Biosynthesis, Sensing and Response, C4 Photosynthesis and Metabolism, Carotenoid Biosynthesis, Chromatin Epigenetics, Heat Shock Proteins and Transcription Factors, Phenylpropanoid Biosynthesis, Photosynthesis related genes, Transporters, Trehalose, WRKY Transcription Factors, and a combination thereof.

In another embodiment, an agrochemical composition disclosed herein can be used for one or more selected from the group consisting of increasing nutrient efficiency, translocation and uptake of nutrients, increasing abiotic stress tolerance, growth stimulation, improving plant health and yield, and a combination thereof of a plant or part thereof.

In another embodiment, disclosed herein is a method for one or more selected from the group consisting of increasing nutrient efficiency, translocation of uptake of nutrients and/or increasing abiotic stress in a plant or part thereof, said method comprising applying to the plant, a part thereof, or a locus, an agrochemical composition as described herein.

In another embodiment, disclosed herein is method for upregulating expression of at least one gene associated with a biochemical or a physiological pathway selected from the group consisting of Amino Acid Transport and Metabolism, Anti-Oxidant Activity, Aquaporin Biosynthesis, Auxin Biosynthesis, Sensing and Response, C4 Photosynthesis and Metabolism, Carotenoid Biosynthesis, Chromatin Epigenetics, Heat Shock Proteins and Transcription Factors, Phenylpropanoid Biosynthesis, Photosynthesis related genes, Transporters, Trehalose, WRKY Transcription Factors, and a combination thereof, in a plant or part thereof, said method comprising applying to the plant, a part thereof, or a locus, an agrochemical composition as described herein; wherein the upregulation of expression of at least one gene results in increase in at least one activity selected from the group consisting of nutrient efficiency, translocation and uptake of nutrients, abiotic stress tolerance and a combination thereof.

In another embodiment, disclosed herein is a method of increasing nutrient use efficiency in a plant, the method comprising applying a composition described herein to the plant under fixed nutrient availability, wherein the composition enhances assimilation and utilization of available nutrients without increasing nutrient input.

In embodiments relating to increasing nutrient use efficiency under fixed nutrient availability, the composition described herein induces differential gene expression in pathways governing nitrogen assimilation and organic nitrogen metabolism. Upregulated genes include those encoding nitrate reductase (NR) and nitrite reductase (NiR), which catalyze the reduction of nitrate to ammonium, as well as glutamine synthetase (GS) and glutamate synthase (GOGAT), which incorporate ammonium into glutamine and glutamate. Differential expression of asparagine synthetase (ASN) and aminotransferases further facilitates nitrogen storage and redistribution. In addition, upregulation of amino acid transporters (e.g., AAP and LHT family genes) supports redistribution of assimilated nitrogen between source and sink tissues.

Collectively, this transcriptional profile enables more efficient assimilation and utilization of available nutrients without increasing nutrient input.

In another embodiment, disclosed herein is a method of improving abiotic and biotic stress physiology in a plant, the method comprising applying a composition described herein to the plant, wherein the composition enhances stress tolerance by maintaining physiological and molecular functionality during periods of environmental stress in the plant.

In embodiments relating to improving abiotic and biotic stress physiology, the composition described herein induces differential gene expression in stress-associated regulatory and protective pathways. This includes modulation of heat shock proteins (HSP20, HSP70) and chaperone-associated genes that regulate proteostasis, together with differential expression of WRKY transcription factors and MAPK signaling components involved in early stress perception. Concurrent upregulation of antioxidant genes encoding superoxide dismutase (SOD), catalase (CAT), and ascorbate peroxidase (APX) enhances detoxification of reactive oxygen species. This coordinated gene expression profile preserves protein functionality, maintains metabolic activity, and improves physiological performance under abiotic and biotic stress conditions.

In another embodiment, disclosed herein is a method for improving translocation of water, sugars, and nutrients within a plant, the method comprising applying a composition described herein to the plant, wherein the composition enhances internal transport of water, photoassimilates, and mineral nutrients between plant tissues.

In embodiments relating to improved translocation of water, sugars, and nutrients, the composition described herein induces differential gene expression of membrane transport and carbohydrate signaling pathways. Upregulated genes include aquaporins from the plasma membrane intrinsic protein (PIP), tonoplast intrinsic protein (TIP), and nodulin-26-like intrinsic protein (NIP) families, which increase permeability to water, urea, carbon dioxide, and other small solutes. Differential expression of ABC transporters, sucrose transporters (SUT/SUC), and SWEET sugar transporters facilitates intracellular trafficking and long-distance phloem transport. In parallel, modulation of trehalose-6-phosphate biosynthesis and signaling genes (e.g., TPS/T6P-associated genes) coordinates source-sink relationships, together enhancing internal transport of water, photoassimilates, and mineral nutrients between plant tissues.

In another embodiment, disclosed herein is a method of enhancing abiotic and biotic stress resilience without sustained growth penalty, the method comprising applying a composition described herein to the plant, wherein plants enter a primed physiological state characterized by transient activation of immune-associated signaling pathways without prolonged suppression of photosynthesis or growth-associated metabolism.

In embodiments relating to enhanced abiotic and biotic stress resilience without sustained growth penalty, the composition described herein induces a primed transcriptional state characterized by transient activation of immune-associated pathways with subsequent resolution. Differential expression of pattern-triggered immunity signaling components, WRKY transcription factors, and jasmonate- and salicylate-associated regulators (e.g., JAZ and NPR-related genes) enables rapid stress response initiation, while recovery is supported by normalization of photosynthesis-associated genes, including photosystem II reaction center genes (psbA, psbD) and electron transport components. This balanced gene expression profile allows stress responsiveness without prolonged suppression of growth-associated metabolism or photosynthetic capacity.

In another embodiment, disclosed herein is a method of growth stimulation in a plant, comprising applying a composition described herein to the plant, wherein the treatment promotes root and shoot development and biomass accumulation under fixed nutrient availability.

In embodiments relating to growth stimulation, the composition described herein induces differential gene expression in pathways governing plant development, metabolism, and biomass accumulation. Upregulated genes include those involved in auxin biosynthesis, transport, and response, such as YUCCA, PIN, and ARF family genes, which promote root and shoot elongation, lateral root formation, and vascular development. Differential expression of cell expansion and cell wall modification genes (including expansions) and ribosomal and translational machinery genes further supports increased protein synthesis and growth. Concurrent enhancement of photosynthesis-related and amino acid metabolism genes provides the energy and building blocks required for sustained biomass accumulation under fixed nutrient availability.

### DETAILED DESCRIPTION

For the purposes of the following detailed description, it is to be understood that the invention may assume various alternative variations and step sequences, except where expressly specified to the contrary. Moreover, other than in any operating examples, or where otherwise indicated, all numbers expressing, for example, quantities of materials/ingredients used in the specification are to be understood as being modified in all instances by the term “about”.

As used herein, the term “about” refers to a measurable value such as a parameter, an amount, a temporal duration, and the like and is meant to include variations of +/−10% or less, specifically variations of +/−5% or less, more specifically variations of +/−1% or less, and even more specifically variations of +/−0.1% or less of and from the particularly recited value, in so far as such variations are appropriate to perform in the disclosure described herein.

Furthermore, it is also to be understood that the value to which the modifier “about” refers is itself specifically disclosed herein.

Recitation of ranges of values are merely intended to serve as a shorthand method of referring individually to each separate value falling within the range, unless otherwise indicated herein, and each separate value is incorporated into the specification as if it were individually recited herein. The endpoints of all ranges are included within the range and independently combinable. As used herein, all numerical values or numerical ranges include integers within such ranges and fractions of the values or the integers within ranges unless the context clearly indicates otherwise. Thus, for example, reference to a range of 90-100%, includes 91%, 92%, 93%, 94%, 95%, 95%, 97%, etc., as well as 91.1%, 91.2%, 91.3%, 91.4%, 91.5%, etc., 92.1%, 92.2%, 92.3%, 92.4%, 92.5%, etc., and so forth. All methods described herein can be performed in a suitable order unless otherwise indicated herein or otherwise clearly contradicted by context.

Thus, before describing the present invention in detail, it is to be understood that this invention is not limited to particularly exemplified process parameters or composition that may of course, vary. It is also to be understood that the terminology used herein is for the purpose of describing embodiments of the invention only and is not intended to limit the scope of the invention in any manner. The use of examples anywhere in this specification including examples of any terms discussed herein is illustrative only, and in no way limits the scope and meaning of the invention or of any exemplified term.

It must be noted that, as used in this specification, the singular forms “a,” “an” and “the” include plural referents unless the content clearly dictates otherwise. The terms “preferred” and “preferably” refer to embodiments of the invention that may afford certain benefits, under certain circumstances.

As used herein, the terms “comprising” “including,” “having,” “containing,” “involving,” and the like are to be understood to be open-ended, i.e., to mean including but not limited to.

The term “composition” in the present specification refers to a final composition resulting as a process of adding together or one after the other more than one component.

The term “weak acid” as used herein refers to any material having a pH range of 3 to 6. It also refers to acids which do not completely dissociate in the solution.

The term “locus” as used herein shall denote the vicinity of the target plant or a part thereof in which growth stimulation is desired. The locus includes the vicinity of the target plant or a part thereof which has either emerged or is yet to emerge. The term “target plant” shall include a multitude of desired crop plants or an individual crop plant growing at a locus. The said locus includes the area, medium and soil where the target plant or a part thereof grows. For simplicity of reference, the target plant is referred synonymously as “plant” in the context of the present invention.

The term “raw material form” as used herein refers to untreated and/or commercially procured form of a nutrient/plant nutrient comprising nitrogen, phosphorus, potassium, or a combination thereof.

The term “amino acid source” as used herein refers to any composition(s) which has concentrated amounts of amino acids or wherein amino acids form a major component of its constitution. Said composition(s) may be naturally derived, such as extracts obtained from animal source, vegetable/plant source or microbial source (bacteria, yeasts, fungi, etc.) Said composition(s) may be synthetically produced. Said composition(s) may comprise one or more or all of the 20 known amino acids, including L-forms, D-forms or racemic mixtures.

The term “amino acids” as used herein are optically active organic compounds containing an amino group and a carboxyl group at the carbon atom. Amino Acids can be derived from vegetable/plant or amino acid enzymatically hydrolyzed by microbial source (bacteria, yeasts, fungi, etc)

The term “oligosaccharide” as used herein refers to a polycation polymer selected from chitin, chitosan, chitosan derivatives, cellulose, and carboxymethyl cellulose or its salts such as sodium salt, chito oligosaccharides, dextran, starch, alginate, karragenans, hyaluronic acid, derivatives, or a combination thereof. Preferably, the oligosaccharide is chitosan.

The term “slow and/or sustained release” as used herein refers to the controlled availability of a nutrient from a nutrient-containing particle or composition over time at a rate slower than readily soluble nutrient forms and/or over a duration sufficient to provide prolonged nutrient availability to a plant. Slow and/or sustained release supports increasing nutrient efficiency by reducing rapid nutrient loss and enabling continued uptake, translocation, assimilation, and utilization of nutrients within the plant, including transport from uptake sites to distal tissues.

Such release may result from physical, chemical, or biological characteristics of the nutrient particle and contributes to improved nutrient distribution, retention, and plant performance under equivalent or reduced nutrient input conditions.

The terms “growth stimulation” or “stimulating growth” as used herein refers to any enhancement of plant health, vigor, development, productivity, or quality relative to an untreated control, carrier-treated control, or baseline condition. Growth stimulation may include initiation, acceleration, or improvement of growth of a plant or any part thereof and may be reflected in enhanced root or shoot growth, improved nutrient use efficiency, increased biomass, improved reproductive development, increased yield or yield components, improved quality of harvested products, improved uniformity or vigor of emergence, enhanced stress tolerance or resistance, delayed senescence, or improved recovery from environmental stress, and may occur in a tissue-specific, systemic, transient, sustained, or temporally staged manner.

The term “differential gene expression” as used herein refers to a change in the level of expression of a gene or set of genes in a plant cell, tissue, organ, or plant relative to a reference level observed in an untreated control, carrier-treated control, or baseline condition, and includes both upregulating gene expression and downregulating gene expression. Upregulating gene expression refers to an increase, and downregulating gene expression refers to a decrease, in gene expression relative to the reference level, which may occur at one or more stages including transcription, RNA accumulation or stability, translation, protein abundance, or enzymatic activity, and may be transient or sustained, tissue-specific or systemic, and temporally staged.

The term “increasing nutrient efficiency” as used herein refers to improving the ability of a plant to acquire, transport, assimilate, utilize, and retain nutrients to support growth, development, and productivity relative to an untreated control, carrier-treated control, or baseline condition, without a corresponding increase in nutrient input. Increasing nutrient efficiency may be evidenced by increased biomass, improved nitrogen status, increased total nutrient content, enhanced nutrient assimilation, reduced nutrient loss, improved yield components, or improved performance under equivalent or reduced nutrient availability, and may result from induced molecular, physiological, or metabolic responses.

The term “translocation of uptake of nutrients” as used herein refers to the combined processes by which a plant absorbs nutrients from its external environment and subsequently transports those nutrients within the plant to support physiological functions. This includes uptake through root or foliar tissues followed by intracellular transport, cell-to-cell movement, and long-distance translocation through vascular tissues to roots, shoots, leaves, reproductive organs, or storage tissues, and may involve modulation of membrane transporters, aquaporins, ion channels, carrier proteins, and vascular transport systems.

The term “increasing abiotic stress” as used herein, when used in the context of plant performance or treatment outcomes, refers to increasing a plant's ability to withstand, tolerate, adapt to, or recover from abiotic stress conditions relative to an untreated control, carrier-treated control, or baseline condition, and is intended to encompass increasing abiotic stress tolerance or resilience rather than increasing the stress itself. Abiotic stress conditions include, but are not limited to, nutrient deficiency, drought, heat, cold, salinity, oxidative stress, and combinations thereof.

The term “improving plant health and yield” as used herein refers to enhancing the overall physiological condition, functional capacity, and productive output of a plant relative to an untreated control, carrier-treated control, or baseline condition. Improving plant health may include enhanced vigor, optimized metabolic function, improved nutrient use efficiency, balanced hormonal regulation, increased stress resilience, and improved recovery from stress, while improving yield may include increases in total or harvestable biomass, reproductive output, yield components, yield quality, or yield stability under equivalent or reduced input conditions.

The term “molecular reprogramming” refers to a coordinated alteration in gene expression, signaling pathways, and downstream metabolic or physiological processes within a plant cell, tissue, or organ, induced by application of an agricultural treatment. Molecular reprogramming includes, but is not limited to, changes in transcriptional activity, signaling cascade activation, transporter regulation, metabolic flux, and hormonal regulation, and results in a functional change in plant behavior relative to untreated controls.

The term “abiotic and biotic stress physiology” as used herein refers to the integrated molecular and physiological responses by which a plant maintains functional performance under abiotic stress conditions and biotic stress conditions. Abiotic stress conditions include, but are not limited to, nutrient deficiency, drought, heat, cold, salinity, and oxidative stress, while biotic stress conditions include stresses arising from pathogens or pests. Abiotic and biotic stress physiology encompasses regulation of stress-associated signaling, antioxidant and redox defense systems, proteostasis and protein stability mechanisms, photosynthetic efficiency, nutrient assimilation, and recovery processes that preserve growth and productivity.

Improvement of abiotic and biotic stress physiology may be reflected in enhanced stress tolerance, maintenance of metabolic and photosynthetic function, and continued growth without chronic stress responses or sustained growth penalties.

An embodiment of the present disclosure provides an agrochemical composition comprising:
a) at least one plant extract;
b) at least one weak acid;
c) an oligosaccharide;
d) an amino acid or an amino acid source; and
e) one or more adjuvant.

In another embodiment disclosed herein, provided is an agrochemical composition comprising:
a) at least one plant extract;
b) at least one weak acid;
c) an oligosaccharide;
d) an amino acid or an amino acid source; and
e) one or more adjuvant;
f) optionally, a nutrient particle or a plurality of nutrient particles, wherein the nutrient particle is a plant nutrient, selected from nitrogen, its compounds or salts; phosphorus, its compounds or salts; potassium, its compounds or salts; or combinations thereof, and
g) optionally, a humic substance selected from humic acid, fulvic acid, or combinations thereof.

In an embodiment, the nutrient particle, wherein the nutrient particle is a plant nutrient, is nitrogen, its compounds or salts. In another embodiment, the nutrient particle is a nitrogenous compound.

In an embodiment, the nutrient particle, wherein the nutrient particle is a plant nutrient, is phosphorus, its compounds or salts. In another embodiment, the nutrient particle is a phosphorous-containing compound.

In an embodiment, the nutrient particle, wherein the nutrient particle is a plant nutrient, is potassium, its compounds or salts. In another embodiment, the nutrient particle is a potassium-containing compound.

In a preferred embodiment, the composition comprises a plurality of nutrient particles, wherein the plurality of nutrient particles is a combination of one or more types of nutrient particles.

In an embodiment, the combinations of nutrient particles comprised in an agrochemical composition described herein may be selected from:
i) a plurality of nutrient particles comprising a nutrient particle comprising nitrogen, its compounds or salts; and phosphorus, its compounds or salts;
ii) a plurality of nutrient particles comprising a nutrient particle comprising potassium, its compounds or salts; and phosphorus, its compounds or salts;
iii) a plurality of nutrient particles comprising a nutrient particle comprising nitrogen, its compounds or salts; and potassium, its compounds or salts;
iv) a plurality of nutrient particles comprising a nutrient particle comprising nitrogen, its compounds or salts; phosphorus, its compounds or salts; and potassium, its compounds or salts.

In a preferred embodiment, the combinations of nutrient particles comprised in an agrochemical composition described herein is a plurality of nutrient particles comprising a nutrient particle comprising nitrogen, its compounds or salts; and phosphorus, its compounds or salts.

In a preferred embodiment, the combinations of nutrient particles comprised in an agrochemical composition described herein is a plurality of nutrient particles comprising a nutrient particle comprising nitrogen, its compounds or salts; phosphorus, its compounds or salts; and potassium, its compounds or salts.

In an embodiment, the nutrient particle comprises nitrogen, its compounds or salts. In an embodiment, the nitrogen-containing nutrient particle is a nitrogen-containing plant nutrient or a nitrogen-containing fertilizer such as those selected from the group consisting of urea (carbamide), ammonium compounds, ammonium fertilizer, urea ammonium nitrate (UAN), cyanamide, guanidine nitrate, dicyandiamide (DCD), guanyl urea sulphate, thiourea, urea formaldehyde polymer, amino acids, amines, amino alcohols, amides, lactams, nitro compounds, imines, nitriles, diazonium salts, hydrazides, carbazides, oximes, alkyl nitrates, nitrosamines, nitroarenes, peroxyacyl nitrates and heterocyclic compounds such as imidazole, triazole, and tetrazole and salts or derivatives thereof.

In a preferred embodiment, the nutrient particle comprising nitrogen/nitrogen-containing plant nutrient/nitrogen-containing fertilizer is urea or salts or derivatives thereof. Examples of derivatives of urea include, but not limited to, N-(2-hydroxyethyl)urea; N-(2-hydroxypropyl)urea; N-(3-hydroxypropyl)urea; N-(2,3-dihydroxypropyl)urea; N-(2,3,4,5,6-pentahydroxyhexyl)urea; N-methyl-N-(1,3,4,5,6-pentahydroxy-2-hexyl)urea; N-methyl-N′-(1-hydroxy-2-methyl-2-propyl)urea; N-(1-hydroxy-2-methyl-2-propyl)urea; N-(1,3-dihydroxy-2-propyl)urea; N-(trishydroxymethylmethyl)urea; N-ethyl-N′-(2-hydroxyethyl)urea; N,N-bis(2-hydroxyethyl)urea; N,N′-bis(2-hydroxyethyl)urea; N,N-bis(2-hydroxypropyl)-urea; N,N′-bis(2-hydroxypropyl)urea; N,N-bis(2-hydroxyethyl)-N′-propylurea; N,N-bis(2-hydroxypropyl)-N′-(2-hydroxyethyl) urea; N-tert-butyl-N′-(2-hydroxyethyl)-N′-(2-hydroxypropyl)urea; N-(1,3-dihydroxy-2-propyl)-N′-(2-hydroxyethyl)urea; N,N-bis(2-hydroxyethyl)-N′,N′-dimethylurea; N,N,N′,N′-tetrakis(2-hydroxyethyl)urea; N′,N′-bis(2-hydroxyethyl)-N′,N′-bis(2-hydroxypropyl)urea, and mixtures thereof.

In a preferred embodiment, the nutrient particle comprising nitrogen/nitrogen-containing plant nutrient/nitrogen-containing fertilizer is an ammonium fertilizer selected from ammonium sulphate, ammonium nitrate, urea ammonium nitrate, anhydrous ammonia, aqueous ammonia, and combinations thereof.

In an embodiment, the nutrient particle comprises nitrogen, its compounds or salts, and phosphorus, its compounds or salts, and is also referred to herein as nitrogen and phosphorous-containing plant nutrient or a nitrogen and phosphorous-containing fertilizer. In a preferred embodiment, the nutrient particle comprising a combination of nitrogen, its compounds or salts, and phosphorus, its compounds or salts is an inorganic ammonium salt selected from ammonium phosphate, diammonium phosphate (DAP), monoammonium phosphate (MAP), and combinations thereof.

In yet another embodiment, the nutrient particle comprising nitrogen, phosphorus, potassium, or a combination thereof is a nitrogen and/or phosphorus and/or potassium containing waste fertilizer, for example, manure or animal excreta, protein mixtures, green manure, fish products, crop residues and other natural materials known to be sources of N or ammonium ions, P or phosphate ions, K or potassium ions, and or various combinations of the foregoing.

In an embodiment, the nutrient particle comprises potassium, its compounds or salts. In an embodiment, the potassium-containing nutrient particle is a potassium-containing plant nutrient or a potassium-containing fertilizer. In an embodiment, the nutrient particle comprising potassium/potassium-containing plant nutrient/potassium-containing fertilizer is potassium chloride, potash or muriate of potash.

In an embodiment, the plant extract belongs to an angiosperm plant or a part thereof. Preferably, in an embodiment, the plant extract is an extract of a plant or a part of plant, wherein the plant is selected from Nelumbonaceae, Musaceae, Rutaceae, Brassicaceae, Moringaceae, Poaceae, Piperaceae, Asphodelaceae, Fabaceae, Phyllanthaceae, Solanaceae, Anacardiaceae, Myrtaceae, Elaeocarpaceae, and combinations thereof. More preferably, in an embodiment, the plant extract is an extract of a plant, or a part of plant selected from the group consisting of genera Nelumbo, Musa, Moringa, Citrus, Brassica, Avena, Cymbopogon, Piper, Tamarindus, Aloe, Phyllanthus, Capsicum, Zea, Mangifera, Solanum, Eucalyptus, Syzygium, Psidium, Elaeocarpus, and combinations thereof. Even more preferably, in an embodiment, the plant extract is an extract of plant, or a part of a plant selected from the group consisting of lotus, banana, moringa, lemon, lime, mustard, oats, betel vine, tamarind, aloe vera, Indian gooseberry (amla), maize, mango, chili, tomato, guava, jamun, plum, rudraksh, eucalyptus, grasses such as lemon grass, and combinations thereof.

In a preferred embodiment, the plant extract is an extract of a plant or a part thereof selected from lotus, banana, and a combination thereof. Preferably, the plant extract is a leaf extract selected from lotus leaf extract, banana leaf extract, and a combination thereof.

In a preferred embodiment, the plant extract is a combination of banana extract or banana leaf extract and plant extract or leaf extracts of lotus, moringa, lemon, lime, mustard, oats, betel vine, tamarind, aloe vera, Indian gooseberry (amla), maize, mango, chili, tomato, guava, jamun, plum, rudraksh, eucalyptus, grasses such as lemon grass.

In a preferred embodiment, the plant extract is an extract of lotus and banana, or parts thereof.

Preferably, the plant extract is a combination of lotus leaf extract and banana leaf extract.

In a preferred embodiment, the plant extract is an extract of betel vine, moringa and banana, or parts thereof. Preferably, the plant extract is a combination of betel vine leaf extract, moringa leaf extract and banana leaf extract.

In an embodiment, the oligosaccharide is a polycation polymer selected from chitin, chitosan, chitosan derivatives, cellulose, and carboxymethyl cellulose or its salts such as sodium salt, chito oligosaccharides, dextran, starch, alginate, karragenans, hyaluronic acid, derivatives, or a combination thereof. Preferably, the oligosaccharide is chitosan.

In an embodiment, a weak acid has a pH range of 3 to 6. In an embodiment, the weak acid is selected from the group consisting of citric acid, malic acid, lactic acid, formic acid, acetic acid, benzoic acid, carbonic acid, and salts or derivatives thereof. In an embodiment, the weak acid is a combination of one or more weak acids or salts or derivatives thereof.

In an embodiment, the composition of the present invention comprises at least one weak acid. In an embodiment, the composition of the present invention comprises at least two weak acids. In one preferred embodiment, the weak acid is citric acid. In another preferred embodiment, the weak acid is malic acid.

In yet another preferred embodiment, the weak acid is malic acid, citric acid, or combinations thereof.

The term “amino acid” comprises one or more of 20 known amino acids. It comprises D-amino acids, L-amino acids, or combinations thereof.

In an embodiment, the amino acid may be an L-amino acid, or derivatives thereof. In an embodiment, the amino acid or L-amino acid is selected from the group consisting of lysine, glycine, aspartic acid, alanine, tryptophan, proline, isoleucine, histidine, leucine, threonine, glutamic acid, tyrosine, serine, glutamine, phenylalanine, cysteine, valine, asparagine, arginine, sarcosine, L-lysine, L-glycine, L-aspartic acid, L-alanine, L-tryptophan, L-proline, L-isoleucine, L-histidine, L-leucine, L-threonine, L-glutamic acid, L-tyrosine, L-serine, L-glutamine, L-phenylalanine, L-cysteine, L-valine, L-asparagine, L-arginine, L-sarcosine, and combinations thereof. In a preferred embodiment, the amino acid or L-amino acid is methionine or L-methionine. In another preferred embodiment, the amino acid or L-amino acid is a combination of one or more amino acids or L-amino acids selected from lysine, glycine, aspartic acid, alanine, tryptophan, proline, isoleucine, histidine, leucine, threonine, glutamic acid, tyrosine, serine, glutamine, phenylalanine, cysteine, valine, asparagine, arginine, sarcosine, L-lysine, L-glycine, L-aspartic acid, L-alanine, L-tryptophan, L-proline, L-isoleucine, L-histidine, L-leucine, L-threonine, L-glutamic acid, L-tyrosine, L-serine, L-glutamine, L-phenylalanine, L-cysteine, L-valine, L-asparagine, L-arginine, L-sarcosine. In an embodiment, the amino acid or L-amino acid is derived from a synthetic source or a natural source. In an embodiment, the amino acid or L-amino acid is derived from microorganisms such as bacteria, fungi, or acid hydrolysis of a cereal such as soy, cereals, quinoa, or the likes.

In an embodiment, the amino acid source is an extract comprising an amino acid.

In an embodiment, the extracts comprising an amino acid selected from microbial extracts, vegetable extracts, or combinations thereof.

In an embodiment, the microbial extract may be a cell-free extract, such as a supernatant of cell lysate or fermentation media without microorganisms. The microbial extract may be live cells or fermentation media comprising microorganisms. In an embodiment, the microbial extract comprises a Bacillus species or an extract of a Bacillus species. In an embodiment, the microbial extract comprises a Pseudomonas species or an extract of a Pseudomonas species.

In a preferred embodiment, the microbial extract is a yeast extract. Yeast extract may or may not comprise live cells or cell debris. Yeast extract comprises amino acids, particularly sulfur-containing amino acids such as methionine and cysteine. In an embodiment, the yeast is selected from the genera Saccharomyces, Kluyveromyces, Hanseniaspora, Metschnikowia, Pichia, Starmerella, Torulaspora, Brettanomyces, Lachancea, Schizosaccharomyces or Candida.

In an embodiment, the vegetable extract source may be leguminous plants, cereals, soy, quinoa, or the likes.

In one embodiment, the present invention provides an agrochemical composition comprising microbial extracts, in particular yeast extract.

In one embodiment, the present invention provides an agrochemical composition comprising amino acid extract, preferably the amino acid extract obtained from a vegetable source.

In an embodiment, the humic substance is selected from humic acid, fulvic acid, or a combination thereof. Preferably, the humic substance is fulvic acid. Preferably, the humic substance is humic acid. Preferably, the humic substance is fulvic acid and humic acid.

The composition of present invention comprises one or more adjuvants selected from co-solvents, surfactants, stabilizers, colorants, dispersants, emulsifiers, thickeners, antifreeze agents, anti-foam agents, wetting agents, solvents, preservatives, viscosity modifiers, penetrants, or a mixture thereof for improved penetration, spreading, and improved viscosity.

In an embodiment, the surfactants may be selected from non-ionic, anionic or cationic surfactants.

Examples of nonionic surfactants include polyarylphenol polyethoxy ethers, polyalkylphenol polyethoxy ethers, polyglycol ether derivatives of saturated fatty acids, polyglycol ether derivatives of unsaturated fatty acids, polyglycol ether derivatives of aliphatic alcohols, polyglycol ether derivatives of cycloaliphatic alcohols, fatty acid esters of polyoxyethylene sorbitan, alkoxylated vegetable oils, alkoxylated acetylenic diols, polyalkoxylated alkylphenols, fatty acid alkoxylates, sorbitan alkoxylates, polyoxyethylene sorbitol, sorbitol esters, polysorbate, C₈-C₂₂ alkyl or alkenyl polyglycosides, polyalkoxy styrylaryl ethers, alkylamine oxides, block copolymer ethers, polyalkoxylated fatty glyceride, polyalkylene glycol ethers, linear aliphatic or aromatic polyesters, organo silicones, polyaryl phenols, sorbitol ester alkoxylates, polyalkylene oxide block copolymers, acrylic copolymers and mono- and diesters of ethylene glycol and mixtures thereof.

Examples of anionic surfactants include alcohol sulfates, alcohol ether sulfates, alkylaryl ether sulfates, alkylaryl sulfonates such as alkylbenzene sulfonates and alkylnaphthalene sulfonates and salts thereof, alkyl sulfonates, mono- or di-phosphate esters of polyalkoxylated alkyl alcohols or alkylphenols, mono- or di-sulfosuccinate esters of C₁₂-C₁₅ alkanols or polyalkoxylated C₁₂-C₁₅ alkanols, alcohol ether carboxylates, phenolic ether carboxylates, polybasic acid esters of ethoxylated polyoxyalkylene glycols consisting of oxybutylene or the residue of tetrahydrofuran, sulfoalkylamides and salts thereof such as N-methyl-N-oleoyltaurate Na salt, polyoxyalkylene alkylphenol carboxylates, polyoxyalkylene alcohol carboxylates alkyl polyglycoside/alkenyl succinic anhydride condensation products, alkyl ester sulfates, napthalene sulfonates, naphthalene formaldehyde condensates, alkyl sulfonamides, sulfonated aliphatic polyesters, sulfate esters of styrylphenyl alkoxylates, and sulfonate esters of styrylphenyl alkoxylates and their corresponding sodium, potassium, calcium, magnesium, zinc, ammonium, alkylammonium, diethanolammonium, or triethanolammonium salts, salts of ligninsulfonic acid such as the sodium, potassium, magnesium, calcium or ammonium salt, polyarylphenol polyalkoxyether sulfates and polyarylphenol polyalkoxyether phosphates, and sulfated alkyl phenol ethoxylates and phosphated alkyl phenol ethoxylates.

Cationic surfactants include alkanol amides of C₈-C₁₈ fatty acids and C₈-C₁₈ fatty amine polyalkoxylates, C₁₀-C₁₈ alkyldimethylbenzylammonium chlorides, coconut alkyldimethylaminoacetic acids, and phosphate esters of C₈-C₁₈ fatty amine polyalkoxylates.

In another embodiment, antifreeze agent(s) added to the composition may be alcohols selected from the group comprising of but not limited to ethylene glycol, 1,2-propylene glycol, 1,3-propylene glycol, 1,2-butanediol, 1,3-butanediol, 1,4-butanediol, 1,4-pentanediol, 3-methyl-1,5-pentanediol, 2,3-dimethyl-2,3-butanediol, trimethylol propane, mannitol, sorbitol, glycerol, pentaerythritol, 1,4-cyclohexanedimethanol, xylenol, bisphenols such as bisphenol A or the like. In addition, ether alcohols such as diethylene glycol, triethylene glycol, tetraethylene glycol, polyoxyethylene or polyoxypropylene glycols of molecular weight up to about 4000, diethylene glycol monomethylether, diethylene glycol monoethylether, triethylene glycol monomethylether, butoxyethanol, butylene glycol monobutylether, dipentaerythritol, tripentaerythritol, tetrapentaerythritol, diglycerol, triglycerol, tetraglycerol, pentaglycerol, hexaglycerol, heptaglycerol, octaglycerol.

In an embodiment, the preservative is selected from 1,2-benzisothiazolin-3-one; dipropylene glycol solution of 1,2-benzisothiazolin-3-one; 2-Methyl-2H-isothiazol-3-one; sodium benzoate; potassium benzoate; benzoic acid; sorbic acid; potassium sorbate; sodium sulphite; potassium sulphite; sodium bisulphite; potassium bisulphite; sodium metabisulphite; potassium metabisulphite; sodium methylparaben; citric acid or salts thereof, malic acid or salts thereof; tartaric acid or salts thereof, and propionic acid or salts thereof.

In an embodiment, the stabilizing agent is a pH stabilizing agent/pH adjuster. In an embodiment, the pH adjuster may be selected from alkaline or acidic pH adjusters. The pH adjuster may be selected from acids or alkali solutions. In an embodiment, the alkali solution is sodium hydroxide and/or potassium hydroxide. In an embodiment, acidic pH adjuster may be a weak acid selected from the weak acids embodied in the specification. In a preferred embodiment, malic acid may be used as a pH adjuster.

The inventors of the present application surprisingly found that the composition disclosed improved Abiotic and Biotic stress physiology in a plant by upregulating heat shock factors to preserve functionality of proteins during periods of stress. Further, it was unexpected to find that the composition disclosed herein improved translocation of sugars, nutrients and water through upregulation of aquaporins and trehalose-6-phosphate. It was both unexpected and surprising that application of the composition described herein impacts molecular expression of genes involved in 1) cellular and subcellular uptake and translocation of water, nutrients, carbon dioxide, photo assimilates and other small molecules, 2) abiotic stress tolerance and 3) regulation of phytohormone biosynthesis, signaling and response.

In an embodiment, the compositions of the present invention are a fertilizer composition.

The compositions as disclosed in the present invention may be present in solid or liquid form.

The compositions may be in form a solid, such as dusts, granules, water dispersible granules, dry flowables, powders, pellets, tablets, microcapsules, micro-complexes, and so forth. The compositions may be in form a liquid, such as a suspension concentrate, an emulsion, an oil dispersion, an emulsifiable concentrate, and so forth. The composition may be in form of a semi-solid, such as a gel, a paste, a cream, and so forth. In a preferred embodiment, the composition is a liquid composition.

In an embodiment, the compositions of the present invention have a pH in the range of about 4 to 9. It was observed that the composition ingredients and process were instrumental in producing a composition with said pH range and thus resulting in a stable, efficient composition. The pH of the composition plays an important role in the product stability, storage and efficacy.

In an embodiment, the nitrogen content in the nutrient particle comprising nitrogen, or a combination of nitrogen and phosphorus ranges from about 5% w/w or w/v to about 85% w/w or w/v of the total weight of the compound.

In an embodiment, the phosphorus content in the nutrient particle comprising phosphorus, or a combination of phosphorus and nitrogen ranges from about 5% w/w or w/v to about 85% w/w or w/v of the total weight of the compound.

In an embodiment, the nitrogen content in the composition of the present invention ranges from about 1% w/w or w/v to about 40% w/w or w/v of the total weight of the composition. In a preferred embodiment, the nitrogen content in the composition of the present invention is in the range of about 4% w/w or w/v to about 6% w/w or w/v of the total weight of the composition.

In an embodiment, the phosphorus content in the composition of the present invention ranges from about 1% w/w or w/v to about 40% w/w or w/v of the total weight of the composition.

In a preferred embodiment, the nitrogen content in the composition of the present invention is in the range of about 4% w/w or w/v to about 6% w/w or w/v of the total weight of the composition.

In an embodiment, the composition comprises the plant extract in an amount ranging from about 5% w/w or w/v to about 90% w/w or w/v of the total weight of the composition. In an embodiment, the composition comprises the plant extract in an amount ranging from about 10% w/w or w/v to about 85% w/w or w/v of the total weight of the composition. In an embodiment, the composition comprises the plant extract in an amount ranging from about 15% w/w or w/v to about 80% w/w or w/v of the total weight of the composition. In an embodiment, the composition comprises the plant extract in an amount ranging from about 20% w/w or w/v to about 75% w/w or w/v of the total weight of the composition. In an embodiment, the composition comprises the plant extract in an amount ranging from about 25% w/w or w/v to about 70% w/w or w/v of the total weight of the composition.

In an embodiment, the composition comprises the oligosaccharide in an amount ranging from about 0.001% w/w or w/v to about 75% w/w or w/v of the total weight of the composition. In an embodiment, the composition comprises the oligosaccharide in an amount ranging from about 0.001% w/w or w/v to about 60% w/w or w/v of the total weight of the composition. In an embodiment, the composition comprises the oligosaccharide in an amount ranging from about 0.001% w/w or w/v to about 50% w/w or w/v of the total weight of the composition.

In an embodiment, the composition comprises the weak acid in an amount ranging from about 0.01% w/w or w/v to about 40% w/w or w/v of the total weight of the composition. In an embodiment, the composition comprises the weak acid in an amount ranging from about 0.01% w/w or w/v to about 30% w/w or w/v of the total weight of the composition. In an embodiment, the composition comprises the weak acid in an amount ranging from about 0.01% w/w or w/v to about 10% w/w or w/v of the total weight of the composition.

In an embodiment, the composition comprises the amino acid in an amount ranging from about 1% w/w or w/v to about 90% w/w or w/v of the total weight of the composition. In an embodiment, the composition comprises the amino acid in an amount ranging from about 10% w/w or w/v to about 70% w/w or w/v of the total weight of the composition. In an embodiment, the composition comprises the amino acid in an amount ranging from about 20% w/w or w/v to about 50% w/w or w/v of the total weight of the composition.

In an embodiment, the composition comprises the adjuvant in an amount ranging from about 1% w/w or w/v to about 70% w/w or w/v of the total weight of the composition. In an embodiment, the composition comprises the adjuvant in an amount ranging from about 5% w/w or w/v to about 50% w/w or w/v of the total weight of the composition. In an embodiment, the composition comprises the adjuvant in an amount ranging from about 10% w/w or w/v to about 40% w/w or w/v of the total weight of the composition.

In an embodiment, the composition comprises the additional stabilizing agent(s) in an amount ranging from about 0.001% w/w or w/v to about 40% w/w or w/v of the total weight of the composition.

In an embodiment, the nitrogen content of the raw material form of nutrient particle is in the range of about 1% w/w to about 80% w/w of the total weight of the compound.

In another embodiment, disclosed herein is an agrochemical composition for upregulating genes associated with a biochemical or physiological pathway in a plant when applied to the plant, a part thereof, or a locus an agrochemical composition described herein.

In another embodiment, disclosed herein is an agrochemical composition for upregulating genes associated with a biochemical or physiological pathway in a plant when applied to the plant, a part thereof, or a locus, an agrochemical composition described herein.

In one aspect of an embodiment disclosed herein, the biochemical or physiological pathway can be selected from the group consisting of Amino Acid Transport and Metabolism, Anti-Oxidant Activity, Aquaporin Biosynthesis, Auxin Biosynthesis, Sensing and Response, C4 Photosynthesis and Metabolism, Carotenoid Biosynthesis, Chromatin—Epigenetics, Heat Shock Proteins and Transcription Factors, Phenylpropanoid Biosynthesis, Photosynthesis related genes, Transporters, Trehalose, WRKY Transcription Factors, and a combination thereof.

In another embodiment disclosed herein, the upregulated genes can be selected from the group consisting of malate dehydrogenase [EC:1.1.1.37]; malate dehydrogenase, cytoplasmic-like, fumarate hydratase, class II [EC:4.2.1.2]; pco106335; uncharacterized protein LOC100279871; (+)-abscisic acid 8′-hydroxylase [EC:1.14.14.137]; putative cytochrome P450 superfamily protein precursor; abscisic-aldehyde oxidase [EC:1.2.3.14]; indole-3-acetaldehyde oxidase, zeaxanthin epoxidase [EC:1.14.15.21]; zeaxanthin epoxidase, (+)-abscisic acid 8′-hydroxylase [EC:1.14.14.137]; umc1299; uncharacterized protein LOC100274595 precursor, 9-cis-epoxycarotenoid dioxygenase [EC:1.13.11.51]; uncharacterized protein LOC100501454, 9-cis-epoxycarotenoid dioxygenase [EC:1.13.11.51]; uncharacterized protein LOC100281391, 15-cis-phytoene desaturase [EC:1.3.5.5]; phytoene dehydrogenase, chloroplastic/chromoplastic, violaxanthin de-epoxidase [EC:1.23.5.1]; violaxanthin de-epoxidase; 9-cis-epoxycarotenoid dioxygenase [EC:1.13.11.51]; uncharacterized protein LOC100281391; catalase [EC:1.11.1.6]; cat2; catalase isozyme 2, L-ascorbate peroxidase [EC:1.11.1.11]; Thylakoid lumenal 29 kDa protein chloroplastic precursor, cytokinin dehydrogenase [EC:1.5.99.12]; CKX10; cytokinin dehydrogenase 10, soluble epoxide hydrolase/lipid-phosphate phosphatase [EC:3.3.2.10 3.1.3.76]; uncharacterized protein LOC100841009, soluble epoxide hydrolase/lipid-phosphate phosphatase [EC:3.3.2.10 3.1.3.76]; uncharacterized protein LOC8071538, L-ascorbate peroxidase [EC:1.11.1.11]; L-ascorbate peroxidase 1, cytosolic; peroxisomal membrane protein 2; Peroxisomal membrane 22 kDa (Mpv17/PMP22) family protein precursor, soluble epoxide hydrolase/lipid-phosphate phosphatase [EC:3.3.2.10 3.1.3.76]; LOW QUALITY PROTEIN: uncharacterized protein LOC111302718, glutathione S-transferase [EC:2.5.1.18]; IDP340; uncharacterized protein LOC100216831, abscisic-aldehyde oxidase [EC:1.2.3.14]; indole-3-acetaldehyde oxidase, glutathione S-transferase [EC:2.5.1.18]; gst1; glutathione S-transferase 1, zeaxanthin epoxidase [EC:1.14.15.21]; zeaxanthin epoxidase, (+)-abscisic acid 8′-hydroxylase [EC:1.14.14.137]; umc1299; uncharacterized protein LOC100274595 precursor, UDP-glucosyltransferase 85A [EC:2.4.1.-]; UDP-glycosyltransferase 85A2 isoform X2, 9-cis-epoxycarotenoid dioxygenase [EC:1.13.11.51]; uncharacterized protein LOC100501454, glutathione S-transferase [EC:2.5.1.18]; gst42; glutathione S-transferase42, 9-cis-epoxycarotenoid dioxygenase [EC:1.13.11.51]; uncharacterized protein LOC100281391, peroxin-5; uncharacterized protein LOC100191477, spermidine synthase [EC:2.5.1.16]; spesyn; spermine synthase1, cytokinin trans-hydroxylase; cytokinin hydroxylase, UDP-glucosyltransferase 85A [EC:2.4.1.-]; UDP-glycosyltransferase 85A5 isoform X1, 15-cis-phytoene desaturase [EC:1.3.5.5]; phytoene dehydrogenase, chloroplastic/chromoplastic, violaxanthin de-epoxidase [EC:1.23.5.1]; violaxanthin de-epoxidase, pyridoxine 4-dehydrogenase [EC:1.1.1.65]; predicted protein, cytokinin dehydrogenase [EC:1.5.99.12]; cko3; cytokinin oxidase 3 precursor, pyridoxine 4-dehydrogenase [EC:1.1.1.65]; uncharacterized protein LOC100193172; L-ascorbate peroxidase [EC:1.11.1.11]; L-ascorbate peroxidase 1, cytosolic, glutathione S-transferase [EC:2.5.1.18]; IDP340; uncharacterized protein LOC100216831, UDP-glucosyltransferase 85A [EC:2.4.1.-]; UDP-glycosyltransferase 85A2 isoform X2, 9-cis-epoxycarotenoid dioxygenase [EC:1.13.11.51]; uncharacterized protein LOC100281391, glutathione S-transferase [EC:2.5.1.18]; csu454(gst); uncharacterized protein LOC100282447, glutathione S-transferase [EC:2.5.1.18]; GST39; glutathione transferase39, monodehydroascorbate reductase (NADH) [EC:1.6.5.4]; monodehydroascorbate reductase 4, peroxisomal-like, glucuronokinase [EC:2.7.1.43]; LOC109763399; probable glucuronokinase 2, ribonucleoside-diphosphate reductase subunit M2 [EC:1.17.4.1]; uncharacterized protein LOC100192012, glutathione S-transferase [EC:2.5.1.18]; IN2-1 protein, aldehyde dehydrogenase (NAD+) [EC:1.2.1.3]; aldehyde dehydrogenase 5, mitochondrial-like, sarcosine oxidase/L-pipecolate oxidase [EC:1.5.3.1 1.5.3.7]; uncharacterized protein LOC100283462, (S)-2-hydroxy-acid oxidase [EC:1.1.3.15]; gol; glycolate oxidase 1; solute carrier family 15 (peptide/histidine transporter), member 3/4; peptide transporter PTR2, indole-3-glycerol-phosphate lyase [EC:4.1.2.8]; indole-3-glycerol phosphate lyase, chloroplastic-like, basic amino acid/polyamine antiporter, APA family; IDP630; Cationic amino acid transporter 1, vacuolar protein sorting-associated protein IST1; hypothetical protein, aminoacylase [EC:3.5.1.14]; aminoacylase-1, glutamine-fructose-6-phosphate transaminase (isomerizing) [EC:2.6.1.16]; uncharacterized protein LOC100194010, 3-methylcrotonyl-CoA carboxylase alpha subunit [EC:6.4.1.4]; methylcrotonoyl-CoA carboxylase subunit alpha, mitochondrial; solute carrier family 15 (peptide/histidine transporter), member 3/4; pco137042; uncharacterized protein LOC100281589, indole-3-glycerol phosphate synthase [EC:4.1.1.48]; indole-3-glycerol phosphate synthase, 5-methyltetrahydropteroyltriglutamatehomocysteine methyltransferase [EC:2.1.1.14]; csu503(met); uncharacterized LOC541942, delta-1-pyrroline-5-carboxylate synthetase [EC:2.7.2.11 1.2.1.41]; AY109740; uncharacterized LOC778431, arogenate/prephenate dehydratase [EC:4.2.1.91 4.2.1.51]; uncharacterized protein LOC100193934, solute carrier family 32 (vesicular inhibitory amino acid transporter); amino acid transporter AVT1I, spermidine synthase [EC:2.5.1.16]; spesyn; spermine synthase1, choline transport protein; choline transport protein-like, glutamine synthetase [EC:6.3.1.2]; gln3; glutamine synthetase root isozyme 5, thiosulfate/3-mercaptopyruvate sulfurtransferase [EC:2.8.1.1 2.8.1.2]; thiosulfate/3-mercaptopyruvate sulfurtransferase 2 isoform X2, malate dehydrogenase [EC:1.1.1.37]; malate dehydrogenase, cytoplasmic-like, formamidase [EC:3.5.1.49]; uncharacterized protein LOC100274074, polyamine oxidase [EC:1.5.3.17 1.5.3.-]; probable polyamine oxidase 2, aminocyclopropanecarboxylate oxidase [EC:1.14.17.4]; ACO31; acc oxidase, isovaleryl-CoA dehydrogenase [EC:1.3.8.4]; uncharacterized protein LOC100382372; solute carrier family 32 (vesicular inhibitory amino acid transporter); amino acid transporter AVT1I, glutamate decarboxylase [EC:4.1.1.15]; glutamate decarboxylase 2, vacuolar protein sorting-associated protein IST1; hypothetical protein, glucuronokinase [EC:2.7.1.43]; LOC109763399; probable glucuronokinase 2, aminoacylase [EC:3.5.1.14]; aminoacylase-1, 2-oxoisovalerate dehydrogenase E1 component alpha subunit [EC:1.2.4.4]; pco079304; uncharacterized protein LOC100191513, 6-phosphofructokinase 1 [EC:2.7.1.11]; ATP-dependent 6-phosphofructokinase 6-like, malate dehydrogenase [EC:1.1.1.37]; malate dehydrogenase, cytoplasmic-like, sarcosine oxidase/L-pipecolate oxidase [EC:1.5.3.1 1.5.3.7]; uncharacterized protein LOC100283462, aldehyde dehydrogenase (NAD+) [EC:1.2.1.3]; aldehyde dehydrogenase 5, mitochondrial-like; sucrose synthase [EC:2.4.1.13]; sus2; sucrose synthase 2, trehalose 6-phosphate synthase/phosphatase [EC:2.4.1.15 3.1.3.12]; gpm600; uncharacterized protein LOC100281927, trehalose 6-phosphate synthase/phosphatase [EC:2.4.1.15 3.1.3.12]; alpha,alpha-trehalose-phosphate synthase [UDP-forming]6, trehalose 6-phosphate synthase/phosphatase [EC:2.4.1.15 3.1.3.12]; uncharacterized protein LOC103654185, malate dehydrogenase (oxaloacetate-decarboxylating)(NADP+) [EC:1.1.1.40]; me4; NADP malic enzyme 4, chaperonin GroEL; ruBisCO large subunit-binding protein subunit beta, chloroplastic, light-harvesting complex II chlorophyll a/b binding protein 1; chlorophyll a-b binding protein 2, light-harvesting complex I chlorophyll a/b binding protein 4; uncharacterized protein LOC100280410, light-harvesting complex II chlorophyll a/b binding protein 4; uncharacterized protein LOC100216729, photosystem II Psb27 protein; photosystem II 11 kD protein, catalase [EC:1.11.1.6]; cat2; catalase isozyme 2, alpha-amylase [EC:3.2.1.1]; alpha-amylase isozyme 2A, glucan endo-1,3-beta-glucosidase 4 [EC:3.2.1.39]; glucan endo-1,3-beta-glucosidase 4-like isoform X2, beta-glucosidase [EC:3.2.1.21]; beta-glucosidase 12, malate synthase [EC:2.3.3.9]; mas1; malate synthase, glyoxysomal, beta-amylase [EC:3.2.1.2]; pco104637; beta-amylase, glucan endo-1,3-beta-glucosidase 1/2/3 [EC:3.2.1.39]; hypothetical protein, inositol 3-alpha-galactosyltransferase [EC:2.4.1.123]; galactinol synthase 1; chaperonin GroEL; ruBisCO large subunit-binding protein subunit beta, chloroplastic-like, chaperonin GroEL; uncharacterized protein LOC100272259, trehalose 6-phosphate synthase/phosphatase [EC:2.4.1.15 3.1.3.12]; uncharacterized protein LOC103654185, malate dehydrogenase [EC:1.1.1.37]; malate dehydrogenase, cytoplasmic-like, trehalose 6-phosphate phosphatase [EC:3.1.3.12]; trehalose-phosphate phosphatase A-like, chaperonin GroEL; ruBisCO large subunit-binding protein subunit beta, chloroplastic, trehalose 6-phosphate synthase/phosphatase [EC:2.4.1.15 3.1.3.12]; probable alpha,alpha-trehalose-phosphate synthase [UDP-forming]9, trehalose 6-phosphate synthase/phosphatase [EC:2.4.1.15 3.1.3.12]; probable alpha,alpha-trehalose-phosphate synthase [UDP-forming]10, trehalose 6-phosphate synthase/phosphatase [EC:2.4.1.15 3.1.3.12]; alpha,alpha-trehalose-phosphate synthase [UDP-forming]1, trehalose 6-phosphate synthase/phosphatase [EC:2.4.1.15 3.1.3.12]; trehalose-6-phosphate synthase, ferredoxin; fdx2; ferredoxin-2, chloroplastic, UDP-glucosyltransferase 85A [EC:2.4.1.-]; UDP-glycosyltransferase 85A2 isoform X2, UDP-glucosyltransferase 85A [EC:2.4.1.-]; UDP-glycosyltransferase 85A5 isoform X1, fumarate hydratase, class II [EC:4.2.1.2]; pco106335; uncharacterized protein LOC100279871, glutamine synthetase [EC:6.3.1.2]; gln3; glutamine synthetase root isozyme 5, hexokinase [EC:2.7.1.1]; hexokinase-3, glycogen phosphorylase [EC:2.4.1.1]; phol; alpha-1,4-glucan phosphorylase, geranylgeranyl diphosphate/geranylgeranyl-bacteriochlorophyllide a reductase [EC:1.3.1.83 1.3.1.111]; geranylgeranyl diphosphate reductase, chloroplastic, light-harvesting complex II chlorophyll a/b binding protein 1; chlorophyll a-b binding protein 2, light-harvesting complex I chlorophyll a/b binding protein 4; uncharacterized protein LOC100280410, light-harvesting complex II chlorophyll a/b binding protein 4; uncharacterized protein LOC100216729, photosystem II Psb27 protein; photosystem II 11 kD protein, alpha-amylase [EC:3.2.1.1]; alpha-amylase isozyme 2A, glucan endo-1,3-beta-glucosidase 4 [EC:3.2.1.39]; glucan endo-1,3-beta-glucosidase 4-like isoform X2, sucrose synthase [EC:2.4.1.13]; sus2; sucrose synthase 2, beta-amylase [EC:3.2.1.2]; pco104637; beta-amylase, trehalose 6-phosphate synthase/phosphatase [EC:2.4.1.15 3.1.3.12]; gpm600; uncharacterized protein LOC100281927, trehalose 6-phosphate synthase/phosphatase [EC:2.4.1.15 3.1.3.12]; alpha,alpha-trehalose-phosphate synthase [UDP-forming]6, glucan endo-1,3-beta-glucosidase 1/2/3 [EC:3.2.1.39]; hypothetical protein, formamidase [EC:3.5.1.49]; uncharacterized protein LOC100274074, alpha-galactosidase [EC:3.2.1.22]; uncharacterized protein LOC100280970 precursor, alpha-galactosidase [EC:3.2.1.22]; uncharacterized protein LOC100191764 precursor; trehalose 6-phosphate phosphatase [EC:3.1.3.12]; probable trehalose-phosphate phosphatase 7, sucrose synthase [EC:2.4.1.13]; sucrose synthase 7, malate dehydrogenase [EC:1.1.1.37]; malate dehydrogenase, cytoplasmic-like, 6-phosphofructokinase 1 [EC:2.7.1.11]; ATP-dependent 6-phosphofructokinase 6-like, light-harvesting complex II chlorophyll a/b binding protein 1; chlorophyll a-b binding protein 2, light-harvesting complex II chlorophyll a/b binding protein 5; Lhcb5-2; chlorophyll a/b-binding apoprotein CP26 precursor, ferredoxin; fdx5; ferredoxin-5, chloroplastic, ferredoxin; fdx2; ferredoxin-2, chloroplastic, UDP-glucosyltransferase 85A [EC:2.4.1.-]; UDP-glycosyltransferase 85A2 isoform X2, glutamate receptor, ionotropic, plant; glutamate receptor 3.1, glucan 1,3-beta-glucosidase [EC:3.2.1.58]; glucan 1,3-beta-glucosidase-like, alpha-amylase [EC:3.2.1.1]; alpha-amylase isozyme 2A, glucan endo-1,3-beta-glucosidase 1/2/3 [EC:3.2.1.39]; glucan endo-1,3-beta-glucosidase 3-like, glycogenin [EC:2.4.1.186]; glycosyl transferase, families GT8+GT64, granule-bound starch synthase [EC:2.4.1.242]; wx1; granule-bound starch synthase 1, chloroplastic/amyloplastic precursor, beta-galactosidase [EC:3.2.1.23]; beta-galactosidase 2, raffinose synthase [EC:2.4.1.82]; probable galactinolsucrose galactosyltransferase 2, aldehyde dehydrogenase (NAD+) [EC:1.2.1.3]; aldehyde dehydrogenase 5, mitochondrial-like, UDP-glucose 4-epimerase [EC:5.1.3.2]; UDP-glucose-4-epimerase, ribulose-bisphosphate carboxylase small chain [EC:4.1.1.39]; ribulose bisphosphate carboxylase small subunit 2, (S)-2-hydroxy-acid oxidase [EC:1.1.3.15]; gol; glycolate oxidase 1; beta-glucosidase [EC:3.2.1.21]; beta-glucosidase 12, peroxidase [EC:1.11.1.7]; gpm233; uncharacterized protein LOC100273479 precursor; caffeoyl-CoA O-methyltransferase [EC:2.1.1.104]; uncharacterized protein LOC107282093, cinnamyl-alcohol dehydrogenase [EC:1.1.1.195]; c14921_1; uncharacterized protein LOC100281336, peroxidase [EC:1.11.1.7]; putative Peroxidase 48, shikimate O-hydroxycinnamoyltransferase [EC:2.3.1.133]; putrescine hydroxycinnamoyltransferase 1 isoform X1, peroxidase [EC:1.11.1.7]; umc2588; uncharacterized protein LOC100286338 precursor, peroxidase [EC:1.11.1.7]; pco073925; peroxidase 42 precursor, cinnamoyl-CoA reductase [EC:1.2.1.44]; LOC109780731; cinnamoyl-CoA reductase 1-like, peroxidase [EC:1.11.1.7]; peroxidase 44, 4-coumarateCoA ligase [EC:6.2.1.12]; pco063262; uncharacterized protein LOC542166; caffeoyl-CoA O-methyltransferase [EC:2.1.1.104]; uncharacterized protein LOC107282093, peroxidase [EC:1.11.1.7]; putative Peroxidase 48, peroxidase [EC:1.11.1.7]; apl; anionic peroxidase precursor, peroxidase [EC:1.11.1.7]; peroxidase 47, peroxidase [EC:1.11.1.7]; uncharacterized protein LOC100216644, peroxidase [EC:1.11.1.7]; peroxidase 50; catalase [EC:1.11.1.6]; cat2; catalase isozyme 2, solute carrier family 15 (peptide/histidine transporter), member 3/4; peptide transporter PTR2, ATP-binding cassette, subfamily C (CFTR/MRP), member 1 [EC:7.6.2.3]; ABC transporter C family member 3, ATP-binding cassette, subfamily A (ABC1), member 3; SmABCA1; ATP-binding cassette transporter, vacuolar protein sorting-associated protein IST1; hypothetical protein, MFS transporter, PHS family, inorganic phosphate transporter; pht2; phosphate transporter protein 2, basic amino acid/polyamine antiporter, APA family; IDP630; Cationic amino acid transporter 1; protein transport protein SEC61 subunit alpha; uncharacterized protein LOC100217058 isoform X1, magnesium transporter; uncharacterized protein LOC100501448 isoform X1, solute carrier family 35, member E3; similar to glucose-6-phosphate/phosphate translocator, thiosulfate/3-mercaptopyruvate sulfurtransferase [EC:2.8.1.1 2.8.1.2]; thiosulfate/3-mercaptopyruvate sulfurtransferase 2 isoform X2, MFS transporter, SP family, sugar:H+ symporter; hexose carrier protein HEX6, 5-methyltetrahydropteroyltriglutamatehomocysteine methyltransferase [EC:2.1.1.14]; csu503(met); uncharacterized LOC541942, solute carrier family 25 (mitochondrial phosphate transporter), member 23/24/25/41; adenine nucleotide transporter BT1, chloroplastic/mitochondrial-like, MFS transporter, SP family, solute carrier family 2 (myo-inositol transporter), member 13; probable inositol transporter 2, metal transporter CNNM; c19255_1; uncharacterized protein LOC100283152, KUP system potassium uptake protein; probable potassium transporter 4, choline transport protein; choline transport protein-like, solute carrier family 15 (peptide/histidine transporter), member 3/4; pco137042; uncharacterized protein LOC100281589, P-type Ca2+ transporter type 2C [EC:7.2.2.10]; calcium-transporting ATPase 5, plasma membrane-type isoform X1, integrin-linked kinase-associated serine/threonine phosphatase 2C [EC:3.1.3.16]; WAT1-related protein At3g18200 isoform X1, MFS transporter, PHS family, inorganic phosphate transporter; inorganic phosphate transporter 1-2-like, ATP-binding cassette, subfamily G (WHITE), member 2, SNQ2; ABC transporter, ATP-binding protein, ATP-binding cassette, subfamily A (ABC1), member 3; ABC transporter A family member 1 isoform X1, solute carrier family 32 (vesicular inhibitory amino acid transporter); amino acid transporter AVT1I; glucuronokinase [EC:2.7.1.43]; LOC109763399; probable glucuronokinase 2, metal transporter CNNM; c19255_1; uncharacterized protein LOC100283152, integrin-linked kinase-associated serine/threonine phosphatase 2C [EC:3.1.3.16]; WAT1-related protein At3g18200 isoform X1, ATP-binding cassette, subfamily B (MDR/TAP), member 1 [EC:7.6.2.2]; uncharacterized protein LOC111781709, P-type Ca2+ transporter type 2C [EC:7.2.2.10]; probable calcium-transporting ATPase 6, plasma membrane-type isoform X1, solute carrier family 32 (vesicular inhibitory amino acid transporter); amino acid transporter AVT1I, ATP-binding cassette, subfamily A (ABC1), member 3; ABC transporter A family member 1 isoform X1, MFS transporter, PHS family, inorganic phosphate transporter; pht1; phosphate transporter protein 1, ATP-binding cassette, subfamily G (WHITE), member 2; ABC transporter G family member 9-like, ATP-binding cassette, subfamily C (CFTR/MRP), member 1 [EC:7.6.2.3]; ABC transporter C family member 3, vacuolar protein sorting-associated protein IST1; hypothetical protein, solute carrier family 45, member 1/2/4; umc2347; sucrose transporter 1; trehalose 6-phosphate synthase/phosphatase [EC:2.4.1.15 3.1.3.12]; alpha,alpha-trehalose-phosphate synthase [UDP-forming]6, trehalose 6-phosphate synthase/phosphatase [EC:2.4.1.15 3.1.3.12]; uncharacterized protein LOC103654185, trehalose 6-phosphate synthase/phosphatase [EC:2.4.1.15 3.1.3.12]; gpm600; uncharacterized protein LOC100281927, inositol 3-alpha-galactosyltransferase [EC:2.4.1.123]; gols2; galactinol synthase 2, inositol 3-alpha-galactosyltransferase [EC:2.4.1.123]; galactinol synthase 1; trehalose 6-phosphate synthase/phosphatase [EC:2.4.1.15 3.1.3.12]; trehalose-6-phosphate synthase, trehalose 6-phosphate synthase/phosphatase [EC:2.4.1.15 3.1.3.12]; alpha,alpha-trehalose-phosphate synthase [UDP-forming]1, trehalose 6-phosphate synthase/phosphatase [EC:2.4.1.15 3.1.3.12]; probable alpha,alpha-trehalose-phosphate synthase [UDP-forming]9, trehalose 6-phosphate synthase/phosphatase [EC:2.4.1.15 3.1.3.12]; probable alpha,alpha-trehalose-phosphate synthase [UDP-forming]10, trehalose 6-phosphate synthase/phosphatase [EC:2.4.1.15 3.1.3.12]; uncharacterized protein LOC103654185; protein brassinosteroid insensitive 1 [EC:2.7.10.1 2.7.11.1]; brassinosteroid LRR receptor kinase-like, auxin response factor; pco123854; uncharacterized protein LOC100384045, calcium-dependent protein kinase [EC:2.7.11.1]; IDP294; uncharacterized protein LOC100273840, somatic embryogenesis receptor kinase 1 [EC:2.7.10.1 2.7.11.1]; somatic embryogenesis receptor-like kinase 3 precursor, auxin-responsive protein IAA; uncharacterized protein LOC100384587, protein brassinosteroid insensitive 1 [EC:2.7.10.1 2.7.11.1]; systemin receptor SR160, cyclin-dependent kinase 12/13 [EC:2.7.11.22 2.7.11.23]; probable serine/threonine-protein kinase At1g54610, LRR receptor-like serine/threonine-protein kinase FLS2 [EC:2.7.11.1]; LRR receptor-like serine/threonine-protein kinase FLS2, kinase [EC:2.7.1.-]; probable serine/threonine-protein kinase PBL19, kinase [EC:2.7.1.-]; PBL1; PBS1-like 1, endo-1,3(4)-beta-glucanase [EC:3.2.1.6]; uncharacterized protein LOC111487562, kinase [EC:2.7.1.-]; probable receptor-like protein kinase At3g55450 isoform X1, serine incorporator 1/3; serine incorporator 3, brassinosteroid insensitive 1-associated receptor kinase 1 [EC:2.7.10.1 2.7.11.1]; BRASSINOSTEROID INSENSITIVE 1-associated receptor kinase 1 isoform X1, protein brassinosteroid insensitive 1 [EC:2.7.10.1 2.7.11.1]; systemin receptor SR160-like, EIX receptor 1/2; leucine-rich repeat receptor protein kinase EMS1-like, GPN-loop GTPase; LOW QUALITY PROTEIN: uncharacterized protein LOC104799061, alpha-amylase [EC:3.2.1.1]; alpha-amylase isozyme 2A, kinase [EC:2.7.1.-]; receptor protein kinase TMK1-like; mitogen-activated protein kinase kinase kinase ANP1 [EC:2.7.11.25]; mitogen-activated protein kinase kinase kinase NPK1-like, coatomer subunit beta′; LOC109749707; serine/threonine-protein kinase StkP-like isoform X1, auxin response factor; pco123854; uncharacterized protein LOC100384045, pto-interacting protein 1 [EC:2.7.11.1]; pto-interacting protein 1-like, serine/threonine-protein kinase PBS1 [EC:2.7.11.1]; serine/threonine-protein kinase PBS1-like isoform X1, serine/threonine-protein kinase ULK2 [EC:2.7.11.1]; serine/threonine-protein kinase ATGla, kinase [EC:2.7.1.-]; receptor-like protein kinase HSL1, senescence-induced receptor-like serine/threonine-protein kinase; senescence-induced receptor-like serine/threonine-protein kinase isoform X2, brassinosteroid insensitive 1-associated receptor kinase 1 [EC:2.7.10.1 2.7.11.1]; BRASSINOSTEROID INSENSITIVE 1-associated receptor kinase 1-like isoform X1, kinase [EC:2.7.1.-]; probable serine/threonine-protein kinase PBL19, GPN-loop GTPase; LOW QUALITY PROTEIN: uncharacterized protein LOC104799061, kinase [EC:2.7.1.-]; PBL1; PBS1-like 1, auxin-responsive protein IAA; IAA24—auxin-responsive Aux/IAA family member, 5′-AMP-activated protein kinase, catalytic alpha subunit [EC:2.7.11.11]; uncharacterized protein LOC100274837; mitogen-activated protein kinase kinase kinase ANP1 [EC:2.7.11.25]; mitogen-activated protein kinase kinase kinase NPK1-like, coatomer subunit beta′; LOC109749707; serine/threonine-protein kinase StkP-like isoform X1, auxin response factor; pco123854; uncharacterized protein LOC100384045, aquaporin PIP; pip2e; aquaporin PIP2-1, pto-interacting protein 1 [EC:2.7.11.1]; pto-interacting protein 1-like, aquaporin PIP; pip2f; aquaporin PIP2-6, serine/threonine-protein kinase PBS1 [EC:2.7.11.1]; serine/threonine-protein kinase PBS1-like isoform X1, serine/threonine-protein kinase ULK2 [EC:2.7.11.1]; serine/threonine-protein kinase ATGla, senescence-induced receptor-like serine/threonine-protein kinase; senescence-induced receptor-like serine/threonine-protein kinase isoform X2, aquaporin TIP; tip1; aquaporin TIP1-1, brassinosteroid insensitive 1-associated receptor kinase 1 [EC:2.7.10.1 2.7.11.1]; BRASSINOSTEROID INSENSITIVE 1-associated receptor kinase 1-like isoform X1, kinase [EC:2.7.1.-]; probable serine/threonine-protein kinase PBL19, GPN-loop GTPase; LOW QUALITY PROTEIN: uncharacterized protein LOC104799061, kinase [EC:2.7.1.-]; PBL1; PBS1-like 1, auxin-responsive protein IAA; IAA24—auxin-responsive Aux/IAA family member, aquaporin PIP; pip2c; aquaporin PIP2-3, 5′-AMP-activated protein kinase, catalytic alpha subunit [EC:2.7.11.11]; uncharacterized protein LOC100274837; probable WRKY transcription factor 52; disease resistance protein RRS1-like; WRKY transcription factor 33; WRKY transcription factor WRKY24-like isoform X1, ATP-dependent RNA helicase DDX42 [EC:3.6.4.13]; DEAD-box ATP-dependent RNA helicase 24 isoform X1, disease resistance protein RPS2; disease resistance protein At4g27190, WRKY transcription factor 33; WRKY53—superfamily of TFs having WRKY and zinc finger domains, WRKY transcription factor 33; probable WRKY transcription factor 33, WRKY transcription factor 1; WRKY transcription factor 1, probable WRKY transcription factor 52; disease resistance protein RRS1-like; hydroxyproline O-galactosyltransferase HPGT [EC:2.4.1.-]; hydroxyproline O-galactosyltransferase HPGT1, disease resistance protein RPS2; disease resistance protein At4g27190, probable WRKY transcription factor 52; disease resistance protein RRS1-like, ATP-dependent RNA helicase DDX42 [EC:3.6.4.13]; DEAD-box ATP-dependent RNA helicase 24 isoform X1, WRKY transcription factor 1; WRKY transcription factor 1-like, WRKY transcription factor 1; WRKY transcription factor 1, WRKY transcription factor 33; probable WRKY transcription factor 26, WRKY transcription factor 33; probable WRKY transcription factor 33; thioredoxin 1; thioredoxin F-type, chloroplastic-like, E3 ubiquitin-protein ligase ATL4 [EC:2.3.2.27]; RING-H2 finger protein ATL5, heat shock 70 kDa protein 1/2/6/8; pco083553; heat shock cognate 70 kDa protein 2, molecular chaperone DnaJ; chaperone protein DnaJ, E3 ubiquitin-protein ligase ATL10/75/76/77/78 [EC:2.3.2.27]; RING-H2 finger protein ATL74, boron transporter; uncharacterized protein LOC4326215, calreticulin; calreticulin-3 precursor, calreticulin; gpm494; uncharacterized protein LOC100382232 precursor, phytepsin [EC:3.4.23.40]; aspartic proteinase oryzasin-1, mitochondrial chaperone BCS1; AAA-ATPase ASD, mitochondrial-like, aspartyl protease family protein [EC:3.4.23.-]; aspartyl protease family protein 2-like, E3 ubiquitin-protein ligase ATL41 [EC:2.3.2.27]; probable E3 ubiquitin-protein ligase ATL44; ATP-dependent Clp protease ATP-binding subunit ClpB; hsp101; heat-shock protein 101, ATP-dependent Clp protease ATP-binding subunit ClpB; uncharacterized protein LOC100217072 isoform X1, calreticulin; crt2; calreticulin precursor, cathepsin D [EC:3.4.23.5]; aspartic proteinase isoform X1, chaperonin GroEL; ruBisCO large subunit-binding protein subunit beta, chloroplastic, chaperonin GroEL; ruBisCO large subunit-binding protein subunit beta, chloroplastic-like, chaperonin GroEL; uncharacterized protein LOC100272259, chaperonin GroES; 10 kDa chaperonin, mitochondrial, cytosolic prostaglandin-E synthase [EC:5.3.99.3]; uncharacterized protein OsI_027940-like, DnaJ homolog subfamily A member 2; dnaJ protein homolog 2, DnaJ homolog subfamily A member 2; dnaJ protein homolog, DnaJ homolog subfamily A member 2; putative dnaJ chaperone family protein, DnaJ homolog subfamily A member 5; predicted protein, DnaJ homolog subfamily B member 4; dnaJ homolog subfamily B member 1 isoform X1, DnaJ homolog subfamily B member 4; dnaJ homolog subfamily B member 4, DnaJ homolog subfamily B member 4; dnaJ protein, DnaJ homolog subfamily B member 4; dnaJ subfamily B member 5, DnaJ homolog subfamily B member 4; uncharacterized protein LOC100382308, E3 ubiquitin-protein ligase ATL10/75/76/77/78 [EC:2.3.2.27]; RING-H2 finger protein ATL78-like, E3 ubiquitin-protein ligase ATL6/9/15/31/42/55 [EC:2.3.2.27]; E3 ubiquitin-protein ligase ATL31, E3 ubiquitin-protein ligase ATL6/9/15/31/42/55 [EC:2.3.2.27]; putative RING zinc finger domain superfamily protein precursor, E3 ubiquitin-protein ligase RNF 115/126 [EC:2.3.2.27]; E3 ubiquitin-protein ligase RDUF1, E3 ubiquitin-protein ligase RNF115/126 [EC:2.3.2.27]; E3 ubiquitin-protein ligase RING1-like, FK506-binding protein 4/5 [EC:5.2.1.8]; 70 kDa peptidyl-prolyl isomerase, FK506-binding protein 4/5 [EC:5.2.1.8]; peptidyl-prolyl cis-trans isomerase FKBP62-like, FK506-binding protein 4/5 [EC:5.2.1.8]; uncharacterized protein LOC100283417, glutaredoxin 3; Grx_C₄—glutaredoxin subgroup I, glutathione S-transferase [EC:2.5.1.18]; gst1; glutathione S-transferase 1, glutathione S-transferase [EC:2.5.1.18]; gst42; glutathione S-transferase42, heat shock 70 kDa protein 1/2/6/8; heat shock 70 kDa protein-like, heat shock 70 kDa protein 1/2/6/8; heat shock protein 1, heat shock 70 kDa protein 1/2/6/8; probable mediator of RNA polymerase II transcription subunit 37c, heat shock 70 kDa protein 4; heat shock 70 kDa protein 15, heat shock 70 kDa protein 4; heat shock 70 kDa protein 4, heat shock 70 kDa protein 4; heat shock 70 kDa protein 8, heat shock protein 90 kDa beta; endoplasmin precursor, heat shock protein 90 kDa beta; shpl1; shepherd-like 1 precursor, heat shock transcription factor, other eukaryote; c130385_1; uncharacterized protein LOC100280318, heat shock transcription factor, other eukaryote; heat shock factor protein 4, heat shock transcription factor, other eukaryote; heat shock factor protein 7, heat shock transcription factor, other eukaryote; heat shock factor protein HSF30, heat shock transcription factor, other eukaryote; heat stress transcription factor B-2c, heat shock transcription factor, other eukaryote; uncharacterized LOC100280131, HSP20 family protein; 16.0 kDa heat shock protein, peroxisomal, HSP20 family protein; 16.9 kDa class I heat shock protein 1, HSP20 family protein; 17.9 kDa class I heat shock protein, HSP20 family protein; 22.0 kDa class IV heat shock protein, HSP20 family protein; class I heat shock protein 3, HSP20 family protein; class II heat shock protein, HSP20 family protein; hsp18a; 17.0 kDa class II heat shock protein, HSP20 family protein; hsp18f, 17.5 kDa class II heat shock protein, HSP20 family protein; hsp22; low molecular weight heat shock protein precursor, HSP20 family protein; TIDP2749; uncharacterized protein LOC100283766, HSP20 family protein; uncharacterized protein LOC100281798, HSP20 family protein; uncharacterized protein LOC100383105, hsp70-interacting protein; Fes1A, hsp70-interacting protein; uncharacterized protein LOC100275241, KDEL-tailed cysteine endopeptidase [EC:3.4.22.-]; hypothetical protein, large proline-rich protein BAG6; uncharacterized protein LOC110600224 isoform X1, molecular chaperone DnaJ; DNAJ heat shock family protein, molecular chaperone HtpG; hsp82; heat shock protein 82, molecular chaperone HtpG; HSP90-2; HSP protein, molecular chaperone HtpG; pco150589a; uncharacterized protein LOC100272368, molecular chaperone HtpG; pco153543(105); uncharacterized protein LOC100384473, oligosaccharyl transferase complex subunit alpha (ribophorin I); dolichyl-diphospho oligosaccharide protein glycosyltransferase 67 kDa subunit precursor, oligosaccharyl transferase complex subunit beta; dolichyl-diphospho oligosaccharide protein glycosyltransferase 48 kDa subunit precursor, peptidyl-prolyl cis-trans isomerase-like 3 [EC:5.2.1.8]; TIDP9202; uncharacterized protein LOC100280578, phytepsin [EC:3.4.23.40]; aspartic proteinase oryzasin-1, pleiotropic regulator 1; uncharacterized protein LOC9311097, protease IV [EC:3.4.21.-]; putative signal peptide peptidase family protein, protein disulfide-isomerase A1 [EC:5.3.4.1]; PDIL1-1; protein disulfide-isomerase precursor, protein disulfide-isomerase A6 [EC:5.3.4.1]; pdi8; protein disulfide isomerase 8 precursor, protein transport protein SEC61 subunit beta; protein transport protein Sec61 subunit beta, RING-H2 zinc finger protein RHA1; E3 ubiquitin-protein ligase RHA1B, serine/threonine-protein kinase PBS1 [EC:2.7.11.1]; serine/threonine-protein kinase PBS1-like, stress-induced-phosphoprotein 1; Hsp70-Hsp90 organizing protein 3, stress-induced-phosphoprotein 1; hsp70-Hsp90 organizing protein, stress-induced-phosphoprotein 1; pco123267; uncharacterized protein LOC100285569, thioredoxin 1; uncharacterized protein LOC606487, transcription factor TGA; transcription factor TGA7, putative, ubiquitin-conjugating enzyme E2 D [EC:2.3.2.23]; ubiquitin-conjugating enzyme E2 28; ATP-dependent Clp protease ATP-binding subunit ClpB; hsp101; heat-shock protein 101, ATP-dependent Clp protease ATP-binding subunit ClpB; uncharacterized protein LOC100217072 isoform X1, calreticulin; calreticulin-like, calreticulin; gpm494; uncharacterized protein LOC100382232 precursor, DnaJ homolog subfamily A member 2; putative dnaJ chaperone family protein, DnaJ homolog subfamily A member 5; predicted protein, DnaJ homolog subfamily B member 11; dnaJ homolog subfamily B member 11-like precursor, DnaJ homolog subfamily B member 4; dnaJ homolog subfamily B member 4, DnaJ homolog subfamily B member 4; dnaJ protein, DnaJ homolog subfamily B member 4; uncharacterized protein LOC100382308, E3 ubiquitin-protein ligase ATL10/75/76/77/78 [EC:2.3.2.27]; RING-H2 finger protein ATL74, E3 ubiquitin-protein ligase ATL10/75/76/77/78 [EC:2.3.2.27]; RING-H2 finger protein ATL78-like, E3 ubiquitin-protein ligase ATL41 [EC:2.3.2.27]; probable E3 ubiquitin-protein ligase ATL44, E3 ubiquitin-protein ligase RNF115/126 [EC:2.3.2.27]; E3 ubiquitin-protein ligase RING1-like, FK506-binding protein 4/5 [EC:5.2.1.8]; uncharacterized protein LOC100283417, glutathione S-transferase [EC:2.5.1.18]; csu454(gst); uncharacterized protein LOC100282447, glutathione S-transferase [EC:2.5.1.18]; GST39; glutathione transferase39, glutathione S-transferase [EC:2.5.1.18]; IN2-1 protein, heat shock 70 kDa protein 1/2/6/8; heat shock 70 kDa protein-like, heat shock 70 kDa protein 1/2/6/8; heat shock cognate 70 kDa protein, heat shock 70 kDa protein 1/2/6/8; heat shock protein 1, heat shock 70 kDa protein 1/2/6/8; pco083553; heat shock cognate 70 kDa protein 2, heat shock 70 kDa protein 1/2/6/8; probable mediator of RNA polymerase II transcription subunit 37c, heat shock 70 kDa protein 4; heat shock 70 kDa protein 14, heat shock 70 kDa protein 4; heat shock 70 kDa protein 15, heat shock 70 kDa protein 4; heat shock 70 kDa protein 4, heat shock protein 90 kDa beta; endoplasmin precursor, heat shock protein 90 kDa beta; shpl1; shepherd-like 1 precursor, heat shock transcription factor, other eukaryote; heat shock factor protein 4, heat shock transcription factor, other eukaryote; heat shock factor protein 7, heat shock transcription factor, other eukaryote; heat shock factor protein HSF30, heat shock transcription factor, other eukaryote; heat stress transcription factor B-2c, heat shock transcription factor, other eukaryote; uncharacterized LOC100280131, HSP20 family protein; 16.9 kDa class I heat shock protein 1, HSP20 family protein; 17.5 kDa class I heat shock protein, HSP20 family protein; 17.9 kDa class I heat shock protein, HSP20 family protein; class I heat shock protein 3, HSP20 family protein; class II heat shock protein, HSP20 family protein; hspl8a; 17.0 kDa class II heat shock protein, HSP20 family protein; hspl8c; 17.8 kDa class II heat shock protein, HSP20 family protein; hsp22; low molecular weight heat shock protein precursor, HSP20 family protein; uncharacterized protein LOC100281798, HSP20 family protein; uncharacterized protein LOC100383105, hsp70-interacting protein; uncharacterized protein LOC100275241, maleylacetoacetate isomerase [EC:5.2.1.2]; gst18; glutathione S-transferase GST 18, molecular chaperone HtpG; hsp82; heat shock protein 82, molecular chaperone HtpG; HSP90-2; HSP protein, molecular chaperone HtpG; pco153543(105); uncharacterized protein LOC100384473, phytepsin [EC:3.4.23.40]; aspartic proteinase oryzasin-1, phytepsin [EC:3.4.23.40]; aspartic proteinase oryzasin-1, protein disulfide-isomerase A6 [EC:5.3.4.1]; pdi8; protein disulfide isomerase 8 precursor, serine/threonine-protein kinase PBS1 [EC:2.7.11.1]; serine/threonine-protein kinase PBS1-like, stress-induced-phosphoprotein 1; hsp70-Hsp90 organizing protein, transcription factor TGA; transcription factor TGA7, putative, ubiquitin-conjugating enzyme E2 O [EC:2.3.2.24]; probable ubiquitin-conjugating enzyme E2 23, xylem cysteine proteinase [EC:3.4.22.-]; hypothetical protein; aquaporin TIP; tip4a; aquaporin TIP4-1; aquaporin PIP; PIP1-4; aquaporin PIP1-3/PIP1-4, aquaporin TIP; tip1; aquaporin TIP1-1, aquaporin PIP; pip1b; aquaporin PIP1-2, aquaporin TIP; tip2a; aquaporin TIP2-1, aquaporin PIP; pip2c; aquaporin PIP2-3, aquaporin NIP; nip2b; aquaporin NIP2-2, aquaporin PIP; probable aquaporin PIP2-7; aquaporin PIP; PIP1-4; aquaporin PIP1-3/PIP1-4, aquaporin PIP; pip2e; aquaporin PIP2-1, aquaporin PIP; pip2f, aquaporin PIP2-6, aquaporin TIP; tip1; aquaporin TIP1-1, aquaporin PIP; pip2c; aquaporin PIP2-3; DNA gyrase subunit A [EC:5.6.2.2]; probable DNA gyrase subunit A, chloroplastic/mitochondrial, lysine-specific demethylase 8 [EC:1.14.11.27]; lysine-specific demethylase JMJ30 isoform X1; lysine-specific demethylase 8 [EC:1.14.11.27]; lysine-specific demethylase JMJ30 isoform X1, [histone H3]-lysine4/36 N-trimethyltransferase SMYD [EC:2.1.1.354 2.1.1.357]; histone-lysine N-methyltransferase ASHR1, coronatine-insensitive protein 1; uncharacterized protein LOC100382859; and a combination thereof.

In another embodiment disclosed herein, is a method for upregulating expression of at least one gene selected from the group consisting of aquaporins, heat shock proteins, WRKY transcription factors, auxins, trehalose 6 phosphate synthase/phosphatase, peroxidases and a combination thereof, said method comprising applying to the plant, a part thereof, or a locus, an agrochemical composition described herein.

In one embodiment, the agrochemical composition described herein upregulates expression of at least one gene selected from the group consisting of aquaporins, heat shock proteins, WRKY transcription factors, auxins, trehalose 6 phosphate synthase/phosphatase, peroxidases and a combination thereofwhen applied to a plant, a part thereof, or a locus.

In another embodiment, the agrochemical composition described herein when applied to a plant, a part thereof, or a locus results in increase in at least one activity selected from the group consisting of nutrient efficiency, translocation and uptake of nutrients, abiotic stress tolerance and a combination thereof.

As such, in one embodiment, the agrochemical composition described herein increases nutrient efficiency in a plant when applied to the plant, a part thereof, or a locus.

In another embodiment, the agrochemical composition described herein increases translocation and uptake of nutrients in a plant when applied to the plant, a part thereof, or a locus.

In another embodiment, the agrochemical composition described a increases abiotic stress tolerance in a plant when applied to the plant, a part thereof, or a locus.

In one embodiment, the instant application discloses is a method for upregulating expression of at least one gene associated with a biochemical or a physiological pathway selected from the group consisting of Amino Acid Transport and Metabolism, Anti-Oxidant Activity, Aquaporin Biosynthesis, Auxin Biosynthesis, Sensing and Response, C4 Photosynthesis and Metabolism, Carotenoid Biosynthesis, Chromatin Epigenetics, Heat Shock Proteins and Transcription Factors, Phenylpropanoid Biosynthesis, Photosynthesis related genes, Transporters, Trehalose, WRKY Transcription Factors, and a combination thereof, said method comprising applying to the plant, a part thereof, or a locus, an agrochemical composition described herein.

In another embodiment, the instant application discloses a method for increasing nutrient efficiency in a plant, said method comprising applying to the plant, a part thereof, or a locus, an agrochemical composition described herein.

In another embodiment, the instant application discloses herein is a method for translocation of uptake of nutrients in a plant, said method comprising applying to the plant, a part thereof, or a locus, an agrochemical composition described herein.

In another embodiment, the instant application discloses a method for increasing abiotic stress in a plant, said method comprising applying to the plant, a part thereof, or a locus, an agrochemical composition described herein.

In another embodiment, the instant application discloses a method for growth stimulation in a plant or a part thereof, said method comprising applying to the plant, a part thereof, or a locus, an agrochemical composition described herein.

In another embodiment, the instant application discloses a method of improving plant health and yield, said method comprising applying to the plant, a part thereof, or a locus, an agrochemical composition described herein.

The plants for which the compositions prepared by the process of the present invention can be used are agronomically useful plants, for example for example vegetable, fruit and cereal crops, and ornamental plants. The agronomically useful plants are angiosperms selected from the group consisting of Apiaceae, Asteraceae, Brassicaceae, Chenopodiaceae, Convolvulaceae, Cucurbitaceae, Fabaceae, Gramineae, Liliaceae, Polygonaceae, Rosaceae, Solanaceae, Poaceae, the Vitaceae. Thus, in an embodiment, the plant is selected from crops, cereals, fruits, vegetables, nuts, vines, nursery plants and flowers. In an embodiment, non-limiting examples of plant include corn, cereals such as rice, wheat, barley, rye, oat, sorghum, millet, triticale, buckwheat, etc.; cotton, soybean, beet, row crops, legumes, grams, sugar cane, tobacco, etc.; oilseeds such as oilseed rape, peanut/groundnut, rapeseed, sunflower, etc.; vegetables: solanaceous vegetables such as eggplant, tomato, pimento, pepper, potato, etc., cucurbit vegetables such as cucumber, pumpkin, zucchini, water melon, melon, squash, gourds, muskmelon, etc., cruciferous vegetables such as radish, white turnip, horseradish, kohlrabi, cabbage, leaf mustard, broccoli, cauliflower, brussels sprouts, kale, etc., asteraceous vegetables such as burdock, crown daisy, artichoke, lettuce, etc, liliaceous vegetables such as green onion, onion, shallot, garlic, and asparagus, ammiaceous vegetables such as carrot, parsley, celery, parsnip, leek, etc., chenopodiaceous vegetables such as spinach, swiss chard, etc., lamiaceous vegetables such as Perilla frutescens, mint, basil, etc.; herbs and spices such as coriander, chamomile, cassia, catnip, clove, cumin, curry, cilantro, cinnamon, cardamom, dill, anise, juniper, lavender, parsley, rosemary, marigold, mustard, nutmeg, fennel, poppy, thyme, vanilla, saffron, poppy, sage, wintergreen, etc.; flowers, foliage plants, turf grasses, fruits: pome fruits such apple, pear, quince, guava, etc, stone fleshy fruits such as peach, plum, nectarine, cherry, apricot, prune, etc., citrus fruits such as orange, lemon, lime, grapefruit, mandarin, malta, kumquat, pummelo, tangerine, tangor, uniq, etc., nuts such as chestnuts, walnuts, hazelnuts, almond, pistachio, cashew nuts, macadamia nuts, pecan nut, cashew nut, hazel nut, pine nut, etc. berries such as caneberry, strawberry, blueberry, cranberry, blackberry, raspberry, coryberry, darrowberry, dewberry, thornless berry, evergreen blackberry, himalayaberry, hullberry, lavacaberry, loganberry, lowberry, lucretiaberry, mammoth blackberry, marionberry, mora, mures deronce, nectarberry, olallieberry, evergreen berry, phenomenalberry, rangeberry, ravenberry, rossberry, dewberry, tayberry, youngberry, zarzamora, aronia berry, currant, elderberry, barberry, gooseberry, honeysuckle, huckleberry, jostaberry, juneberry, lingonberry, salal, seabuckthorn, bayberry, buffaloberry, chokecherry, maypop, mulberry, bearberry, bilberry, cloudberry, muntries, partridgeberry, etc., grape, kaki fruit, kiwi fruit, olive, plum, banana, coffee, date palm, coconuts, papaya, persimmon, avocado, dragon fruit, pomegranate, lychee, jackfruit, pineapple, passionfruit, sapota, etc., trees other than fruit trees; tea, mulberry, flowering plant, trees such as ash, birch, dogwood, eucalyptus, Ginkgo biloba, lilac, maple, Quercus, poplar, Judas tree, Liquidambar formosana, plane tree, zelkova, 34olocasi arborvitae, fir wood, hemlock, juniper, Pinus, Picea, and Taxus cuspidate, etc., other crops such as chive, day lily, Elegans hosta, Fritillaria, gojiberry, okra, pea, hops, beans, guar, radish, amaranth, jute, fenugreek, lentils, chickpea, artichoke, rhubarb, licorice, sweet potato, Dioscorea japonica, olocasia, ornamental grasses (lawn turf, sod, etc.), varieties and cultivars thereof.

The compositions described herein as per certain embodiments can be applied pre-emergence, post-emergence, before sowing, and/or pre-harvest

In an embodiment, the composition is applied before planting/sowing; at the time of sowing, germination, early leaf stage, early bloom stage, pre-bloom stage, full bloom stage, petal fall stage, swollen bud stage, bud break stage, post petal fall stage, post bud break stage, pre-bud break stage, full pistillate stage, post pollination stage, square formation stage, pegging stage, post-pegging stage, fruit set stage, early mid-bloom, greenup (post-dormancy) stage, bud set stage, etc; after flowering stage; harvest stage; and/or post-harvest. In an embodiment, the compositions of the present invention may be applied anytime throughout the growth stage as conventionally known to a person skilled in the art. The time of application depends on the plant or a part thereof to which the compositions of the present invention are applied; environmental conditions such as nutrient deficiency, biotic and abiotic stresses; type of application and the expected outcome or any other parameter known to a person skilled in the art. Applications can be made throughout the growth of the plant, one or more times a week.

In an embodiment, the composition is applied directly and/or indirectly to the plant and/or to plant propagation material by drenching the soil, by drip application onto the soil, by soil injection, by dipping or by treatment of seeds.

The compositions may be applied by dusting, spraying, granular application, seed pelleting/seed dressing, broadcasting, in furrow application, side dressing, spot application, ring application, root zone application, pralinage, seedling root dip, sett treatment, trunk/stem injection, padding, swabbing, root feeding, soil drenching, capsular placement, baiting, fumigation, banding, foliar application, basal application, space treatment, enclosed space fumigation and such other methods which may help prevent or control or eradicate the disease.

In a preferred embodiment, the compositions described herein are applied as a drench application, in-furrow application, soil, drip irrigation, soil injection, hydroponic application, capillary action application, root infiltration, or a foliar application. In an embodiment, the compositions of the present invention is applied as a seed treatment such as seed soak application, seed coating application, germination treatment; or a rooting/shooting dip treatment. The type of application is decided as per the plant or a part thereof to which the compositions of the present invention are applied, the outcome expected, and the time of application and any other conventional parameters as known to a person skilled in the art.

The present compositions can be applied by the use of conventional ground sprayers, granule applicators, watering (drenching), drip irrigation, spraying, atomizing, broadcasting, dusting, foaming, spreading-on, aerial methods of spraying, aerial methods of application, methods utilizing application using modern technologies such as, but not limited to, drones, robots, predosage device, a knapsack sprayer, a spray tank, a spray plane, or an irrigation system and by other conventional means known to those skilled in the art.

In an embodiment, the compositions described herein may be combined with or applied in association with one or more additional agrochemically active agents. The agrochemically active substance is selected from, but not limited to, fertilizers, mycorrhiza, micronutrients, macronutrients, acaricides, algicides, antifeedants, avicides, bactericides, bird repellents, chemosterilants, fungicides, herbicide safeners, herbicides, insect attractants, insect repellents, insecticides, mammal repellents, mating disruptors, molluscicides, nematicides, plant activators, plant-growth regulators, rodenticides, synergists, virucides, derivatives thereof, biological control agents, superabsorbent polymers and mixtures thereof.

In an embodiment, the compositions described herein may be combined with additional compositions of other macronutrients and micronutrients such as phosphorus, potassium, sulphur, metals, and minerals.

In an embodiment, the compositions described herein are used for slow and sustained release of the nutrient particle. In an embodiment, the compositions prepared by the process of the present invention are used for quick release of the nutrient particle.

In an embodiment, the instant application provides a method of improving plant performance, comprising applying an agricultural treatment to a plant, wherein the treatment comprises a component that functions as a primary elicitor to induce molecular reprogramming, resulting in increased plant size, increased nitrogen status, and increased nitrogen content relative to an untreated plant.

In an embodiment, the instant application provides a method of inducing component-driven molecular reprogramming in a plant, comprising applying an agricultural treatment in which chitosan functions as a primary functional ingredient that enhances growth and nitrogen accumulation independent of other formulation components.

In an embodiment, the instant application provides a method of improving nitrogen status of a plant under fixed nitrogen supply, comprising applying an agricultural treatment that enhances nitrogen assimilation efficiency without increasing nitrogen input.

In an embodiment, the instant application provides a method of enhancing nitrogen use efficiency in a plant, comprising applying an agricultural treatment that increases tissue nitrogen concentration through induced physiological reprogramming rather than nutrient delivery.

In an embodiment, the instant application provides a method of increasing total nitrogen content in a plant, comprising applying an agricultural treatment that coordinately enhances biomass accumulation and nitrogen assimilation, thereby increasing total nitrogen per plant.

In an embodiment, the instant application provides a method of improving nitrogen accumulation efficiency in a plant, comprising applying an agricultural treatment that increases nitrogen content under equivalent nitrogen availability relative to an untreated plant.

In an embodiment, the instant application provides a method of inducing root-dominant molecular reprogramming in a plant, comprising applying an agricultural treatment to aerial tissue such that transcriptomic responses are initiated in root tissue prior to shoot tissue.

In an embodiment, the instant application provides a method of systemically reprogramming plant physiology, comprising foliar application of an agricultural treatment that induces early molecular responses in roots through shoot-to-root signaling.

In an embodiment, the instant application provides a method of inducing temporally staged molecular responses in a plant, comprising applying an agricultural treatment that produces an early signaling phase, an intermediate metabolic coordination phase, and a late stabilization phase.

In an embodiment, the instant application provides a method of inducing controlled molecular reprogramming in a plant, comprising applying an agricultural treatment that initiates transient defense signaling followed by restoration of growth-associated metabolic pathways.

In an embodiment, the instant application provides a method of enhancing nutrient transport in a plant, comprising applying an agricultural treatment that induces coordinated modulation of transporter networks responsible for transmembrane and long-distance transport.

In an embodiment, the instant application provides a method of improving hydraulic and nutrient flux in a plant, comprising applying an agricultural treatment that modulates aquaporin expression across root and shoot tissues.

In an embodiment, the instant application provides a method of inducing plastid reprogramming in root tissue, comprising applying an agricultural treatment that upregulates plastid-associated metabolic and energy-related pathways in roots.

In an embodiment, the instant application provides a method of improving cellular energy balance in a plant, comprising applying an agricultural treatment that alters root plastid function to support nutrient assimilation and growth.

In an embodiment, the instant application provides a method of regulating hormonal balance in a plant, comprising applying an agricultural treatment that increases auxin-associated signaling while reducing cytokinin biosynthesis, thereby enhancing root-driven nutrient acquisition.

In an embodiment, the instant application provides a method of coordinating plant growth and nutrient uptake, comprising applying an agricultural treatment that modulates auxin and cytokinin pathways to favor nutrient use efficiency.

In an embodiment, the instant application provides a method of enhancing stress resilience in a plant without sustained growth penalty, comprising applying an agricultural treatment that transiently activates stress-associated signaling and subsequently restores growth-associated metabolism.

In an embodiment, the instant application provides a method of inducing immune priming in a plant, comprising applying an agricultural treatment that activates defense-associated molecular pathways without constitutive defense activation.

In an embodiment, the instant application provides a method of inducing molecular reprogramming in a plant using an oligosaccharide-based elicitor, comprising applying chitosan to the plant to induce nutrient assimilation, transporter regulation, and growth enhancement.

In an embodiment, the instant application provides a method of improving nitrogen efficiency and plant growth, comprising applying chitosan as a stand-alone agricultural treatment in the

In an embodiment, the instant application provides a method of improving nitrogen efficiency in a plant, comprising applying an agricultural treatment that excludes a plant extract while retaining molecular reprogramming and nitrogen accumulation benefits.

In an embodiment, the instant application provides a method of enhancing plant performance through selective component exclusion, comprising applying an agricultural treatment formulated without a component that negatively affects nitrogen status or nitrogen content.

### EXAMPLES

### Example 1: Agricultural Compositions

Agricultural compositions, Formulation 1, Formulation 2, and Formulation 3 are illustrated in Table 1.1, Table 1.2, and Table 1.3, respectively.

TABLE 1.1
An agrochemical composition comprising Formulation 1
Formulation 1

| | Conc % |
|---|---|
| Components | (w/v) |
| Plant extract | 58.3 |
| Citric acid | 1.097 |
| Chitosan | 0.003 |
| Yeast extract | 29.4 |
| Adjuvants | 11.2 |
| Total | 100 |

TABLE 1.2
An agrochemical composition comprising Formulation 2
Formulation 2

| | Conc % |
|---|---|
| Components | (w/v) |
| Plant extract | 58.3 |
| Citric acid | 1.097 |
| Chitosan | 0.003 |
| Yeast extract | 29.4 |
| Urea | 11.0 |
| Adjuvants | 0.2 |
| Total | 100 |

TABLE 1.3
An agrochemical composition comprising Formulation 3
Formulation 3

| | Conc % |
|---|---|
| Components | (w/v) |
| Plant extract | 40.097 |
| Citric acid | 1.15 |
| Chitosan | 0.003 |
| Amino acid | 31.5 |
| Diammonium phosphate | 27 |
| Adjuvants | 0.25 |
| Total | 100 |

### Example 2: Molecular Gene Expression in Ear Meristematic Tissue

### Study Design and Horticultural Methods

The study was conducted using plastic nursery pots, each containing approximately 11 liters of substrate. The chosen substrate was Berger's BM2 custom blend without added nitrogen, phosphorus, or potassium. The pots were arranged over three metal greenhouse benches in a randomized complete block design. The greenhouse compartment used for the study was equipped with LED lighting set to a 16-hour photoperiod. The temperature thresholds for the compartment were set to 23° C. during the day and 28° C. at night for the first week. Upon the start of the second week the daytime temperature threshold was increased to 26° C. during the day and 21° C. at night. At the start of week three thresholds were increased to 29° C. during the day and 24° C. at night. The thresholds remained at those settings for the remainder of the study.

The compartment utilized water misters to maintain 65% relative humidity.

The study was designed to evaluate the molecular gene expression in ear meristematic tissue after the foliar application of four test products at three timepoints and in a single nitrogen environment. Table 2, below, shows the nitrogen levels, test products, application rates, and harvest timepoints for both shoot and root tissue samples. (n=4) The urea was titrated to provide the same amount of nitrogen as was formulated in Formulation 2. Formulation 1 is formulated without nitrogen.

TABLE 2

| | | Application | | |
|---|---|---|---|---|
| | Product | Rate | | Timepoints |
| Sufficient N | Water | 400 | L/Ha | 4, 24, and 96 |
| | Urea | 0.68 | Kg/Ha | hours after |
| | Formulation 1 | 6 | L/Ha | application |
| | Formulation 2 | 6 | L/Ha | |
| | Formulation 3 | 6 | L/Ha | |
| Mild N | Water | 400 | L/Ha | 4, 24, and 96 |
| Deficiency | Urea | 0.68 | Kg/Ha | hours after |
| | Formulation 1 | 6 | L/Ha | application |
| | Formulation 2 | 6 | L/Ha | |
| | Formulation 3 | 6 | L/Ha | |

The pots were watered to saturation prior to sowing with commercially available germplasm with a relative maturity of 115 days. The seed was treated with conventional seed treatments.

Starting 5 days after sowing, the emerging plants were given a nitrogen-free Hoagland's nutrient solution prepared at 25% strength. At 10 days after sowing, the plants were given the same nutrient solution prepared at 50% strength. At 18 days after sowing, 8 mM ammonium nitrate was added to the 50% strength nutrient solution. The study was designed to have a single nitrogen environment in which the plants were supplied a sufficient level of nitrogen.

At the V6 growth stage, the test products were applied by foliar application. A fabricated boom sprayer equipped with 6 Teejet 11002 VS nozzles was positioned approximately 18 inches above the plant canopy.

After test products were applied, the plants were returned to their original greenhouse location prior to sampling.

### Sample Collection

The ear meristematic tissue was collected from the base of the 6^{th} leaf, counting from the round leaf Due to the small size of the target tissue, each sample pooled ear meristematic tissue from 10 test plants. Using a scalpel, the ear meristematic tissue was isolated and removed from the test plants and placed in a 2 mL centrifuge tube before being submerged in liquid nitrogen. All samples were flash-frozen within two minutes of collection. Samples were kept in liquid nitrogen for at least 15 minutes before being transferred to a −80° C. freezer for storage prior to analysis. The samples were shipped on dry ice to a transcriptomics lab for analysis.

### Sample Analysis

A total of 36 samples were processed for transcriptome sequencing, generating 256.48 Gb Clean Data. At least 6.24 Gb clean data were generated for each sample with minimum 92.81% of clean data achieved quality score of Q30.

Clean reads of each sample were mapped to specified reference genome. Mapping ratio ranged from 81.43% to 90.29%. Prediction of alternative splicing, gene structure optimization analysis and novel gene discovery was processed on top of mapping results, during which 12,174 were discovered and 6,516 novel genes were annotated with a putative function.

In this study, differentially expressed genes (DEGs) were identified using the criteria of Fold Change≥1.5 and Pvalue<0.01. Then GO/KEGG enrichment, GSEA, differential alternative splicing (AS) and protein interactions of DEGs were analyzed.

### RNA Quality Assessment

Purity, concentration and integrity of RNA sample were examined by NanoDrop, Qubit 2.0, Agilent 2100, etc. Only RNA with good quality could move on to following procedures.

### Library Construction

Qualified RNA were processed for library construction. The procedures are described as follow:
(1) mRNA was isolated by Oligo(dT)-attached magnetic beads.
(2) mRNA was then randomly fragmented in fragmentation buffer.
(3) First-strand cDNA was synthesized with fragmented mRNA as template and random hexamers as primers, followed by second-strand synthesis with addition of PCR buffer, dNTPs, RNase H and DNA polymerase I. Purification of cDNA was processed with AMPure XP beads.
(4) Double-strand cDNA was subjected to end repair. Adenosine was added to the end and ligated to adapters. AMPure XP beads were applied here to select fragments within size range of 300-400 bp.
(5) cDNA library was obtained by certain rounds of PCR on cDNA fragments generated from step 4.

### Library Quality Control

In order to ensure the quality of library, Qubit 2.0 and Agilent 2100 were used to examine the concentration of cDNA and insert size. Q-PCR was processed to obtain a more accurate library concentration. Library with concentration larger than 2 nM is acceptable.

### Sequencing

The qualified library was sequenced by the high-throughput sequencing platform with PE150 mode. Clean data with high quality was obtained by filtering Raw data, which removes adapter sequence and reads with low quality. These clean data were further mapped to pre-defined reference genome generating mapped data. Assessment on insert size and sequencing randomness were processed on mapped data as library quality control. Basic analysis on mapped data included gene expression quantification, alternative splicing analysis, novel genes prediction and genes structure optimization.

### Sequencing Quality Control

To ensure the quality of the reads before moving onto analysis, raw data containing data such as primers, adapters, etc., were removed before analysis. Procedures for data quality control were listed as follow:
(1) Trim adapter contaminations
(2) Remove nucleotides with low Quality-score.

### Data Alignment to Reference Genome

The reference genome was pre-defined for the analysis. Version information of the reference genome is: Zea_mays.Zm_B73_REFERENCE_NAM_5.0.genome.fa.

### Novel Gene Discovery

In order to optimize the annotation information of a genome, discovery of novel transcripts and genes were achieved by StringTie on base of reference genome. The mapped reads were assembled and compared with original annotations of the genome. The transcript regions without annotation obtained by above processes are defined as novel transcripts. Excluding short transcripts (coding peptides with less than 50 amino acids) or those containing only one exons, 12,174 novel genes were discovered in this study.

### Functional Annotation of Novel Genes

Novel genes were annotated by DIAMOND against databases including NR, Swiss-Prot, COG, KOG and KEGG. KEGG Orthology of novel genes were obtained by above processes. GO Orthology of novel genes were obtained by the underlying software InterProScan basic on the InterPro database. The amino acid sequences of novel genes were blasted against Pfam database by HMMER to gain the annotation information.

### Differential Expression Analysis

The expression of a gene can be influenced by both external stimuli and internal environment, which is highly temporal-specific and tissue-specific. The genes expressed significantly different under different conditions, such as treatment vs control, wild type vs mutants, different time points, different tissue, etc., are defined as Differentially Expressed Genes (DEG). Similarly, transcripts with significantly different expression level are named Differentially Expressed Transcript (DET). The collection of genes acquired in differential expression analysis is defined as DEG set. In result files, the gene sets were named as “A_VS_B” to specify the comparing pair. Normally, “A” represents control group, wild type or former time point. “B” normally represents corresponding treated group, mutant or later time point. The genes with a higher expression level in B than A are defined as up-regulated genes.

The ones with lower expression level in B are defined as down-regulated genes. Therefore, up-reg and down-reg are relative definitions, which relies on the order of A and B.

Criteria for differentially expressed genes were set as Fold Change (FC)≥1.5 and pvalue<0.01.

Fold change (FC) refers to the ratio of gene expression in two samples. False Discovery Rate (FDR) refers to adjusted p-value, which is used to measure significancy of difference.

### GO Analysis on DEGs

GO (Gene Ontology) database is a structured biological annotation system established in 2000 containing a standard vocabulary of gene and gene products functions. GO annotation system is a directed acyclic graph containing three main branches: Biological Process, Molecular Function and Cellular Component.

### GO Enrichment Analysis on DEGs

DEGs were then subjected to functional enrichment analysis and the enriched GO terms and corresponding inclusion relationships were shown in the directed acyclic graph.

### KEGG Annotation of Differentially Expressed Genes

In biological organisms, series of gene products are working synergistically to perform biological functions, which is so called pathway. Annotating genes within pathway networks could largely benefit further analysis on biological functions. KEGG (Kyoto Encyclopedia of Genes and Genomes) is one of the major databases on pathways, including metabolic pathways of carbohydrates, nucleotides, amino acids and biological degradation of organics. Besides metabolic pathways, KEGG contains comprehensive description on enzymes involved in the pathways, including amino acid sequences, links to PDB database, etc.

### KEGG Pathway Enrichment Analysis on DEGs

In this study, we examined if the pathways are over-presented with DEGs. Enrichment factors and fisher test were applied in the determination of enrichment degree and significancy of the pathway.

Data obtained from the above studies is shown in Table 4.

It is evident from the data that the application of composition disclosed herein increases expression of genes associate with biochemical or physiological pathway is selected from the group consisting of Amino Acid Transport and Metabolism, Anti-Oxidant Activity, Aquaporin Biosynthesis, Auxin Biosynthesis, Sensing and Response, C4 Photosynthesis and Metabolism, Carotenoid Biosynthesis, Chromatin—Epigenetics, Heat Shock Proteins and Transcription Factors, Phenylpropanoid Biosynthesis, Photosynthesis related genes, Transporters, Trehalose, WRKY Transcription Factors, and a combination thereof.

### Example 3a: Molecular Gene Expression in Shoot and Root Tissue

### Study Design and Horticultural Methods

The study was conducted using plastic nursery pots, each containing approximately 11 liters of substrate. The chosen substrate was Berger's BM2 custom blend without added nitrogen, phosphorus, or potassium. The pots were arranged over two metal greenhouse benches in a randomized complete block design. The greenhouse compartment used for the study was equipped with LED lighting set to a 16-hour photoperiod. The temperature thresholds for the compartment were set to 23° C. during the day and 28° C. at night for the first week. Upon the start of the second week the daytime temperature threshold was increased to 26° C. during the day and 21° C. at night. At the start of week three thresholds were increased to 29° C. during the day and 24° C. at night. The thresholds remained at those settings for the remainder of the study.

The compartment utilized water misters to maintain 65% relative humidity.

The study was designed to evaluate the molecular gene expression in shoot and root tissue after the foliar application of four test products at three timepoints and in two differing nitrogen environments. Table 3, below, shows the nitrogen levels, test products, application rates, and harvest timepoints for both shoot and root tissue samples. (n=4) The urea was titrated to provide the same amount of nitrogen as was formulated in Formulation 2. Formulation 1 is the formulated without nitrogen.

TABLE 3

| | | Application | | |
|---|---|---|---|---|
| | Product | Rate | | Timepoints |
| Sufficient N | Water | 400 | L/Ha | 4, 24, and 96 |
| | Urea | 0.68 | Kg/Ha | hours after |
| | Formulation 1 | 6 | L/Ha | application |
| | Formulation 2 | 6 | L/Ha | |
| | Formulation 3 | 6 | L/Ha | |
| Mild N | Water | 400 | L/Ha | 4, 24, and 96 |
| Deficiency | Urea | 0.68 | Kg/Ha | hours after |
| | Formulation 1 | 6 | L/Ha | application |
| | Formulation 2 | 6 | L/Ha | |
| | Formulation 3 | 6 | L/Ha | |

The pots were watered to saturation prior to sowing with commercially available germplasm with a relative maturity of 115 days. The seed was treated with conventional seed treatments.

Starting 5 days after sowing, the emerging plants were given a nitrogen-free Hoagland's nutrient solution prepared at 25% strength. At 10 days after sowing, the plants were given the same nutrient solution prepared at 50% strength. At 18 days after sowing, 8 mM ammonium nitrate was added to the 50% strength nutrient solution. The study was designed to contain two nitrogen environments, one that supplied nitrogen to a sufficient level and another that induced a mild nitrogen deficiency. The nitrogen deficiency was inflicted by applying a lower volume of the ammonium nitrate nutrient solution to the mild n deficient plants, the difference in volume was replaced by water.

At the V6 growth stage, the test products were applied by foliar application. A fabricated boom sprayer equipped with 6 Teejet 11002 VS nozzles was positioned approximately 18 inches above the plant canopy.

After test products were applied, the plants were returned to their original greenhouse location prior to sampling.

### Sample Collection

The samples of shoot tissue were collected from the middle of the youngest mature leaf, removing the mib rib. The shoot tissue, approximately 0.5 by 0.5 inches, was rolled and placed in a 2 mL centrifuge tube before being submerged in liquid nitrogen. The root samples consisted of seminal root tissue taken from the middle of the root structure. Approximately 1 gram of root tissue was collected and rinsed in water to remove excess substrate and then rinsed twice in phosphate-buffered saline (PBS) prior to being placed in a centrifuge tube and flash-frozen in liquid nitrogen. All samples were flash-frozen within two minutes of collection. Samples were kept in liquid nitrogen for at least 15 minutes before being transferred to a −80° C. freezer for storage prior to analysis. Samples were shipped on dry ice to a transcriptomics lab for analysis.

### Sample Analysis

A total of 196 were processed for transcriptome sequencing, generating 1328.50 Gb Clean Data. At least 6.21 Gb clean data were generated for each sample with minimum 89.84% of clean data achieved quality score of Q30.

Clean reads of each sample were mapped to specified reference genome. Mapping ratio ranged from 78.90% to 92.97%. Prediction of alternative splicing, gene structure optimization analysis and novel gene discovery was processed on top of mapping results, during which 23,552 were discovered and 11,962 novel genes were annotated with a putative function.

In this study, differentially expressed genes (DEGs) were identified using the criteria of Fold Change≥1.5 and False Discovery Rate<0.01. Then GO/KEGG enrichment, GSEA, differential alternative splicing (AS) and protein interactions of DEGs were analyzed.

### RNA Quality Assessment

Purity, concentration and integrity of RNA sample were examined by NanoDrop, Qubit 2.0, Agilent 2100, etc. Only RNA with good quality could move on to following procedures.

### Library Construction

Qualified RNA were processed for library construction. The procedures are described as follow:
(1) mRNA was isolated by Oligo(dT)-attached magnetic beads.
(2) mRNA was then randomly fragmented in fragmentation buffer.
(3) First-strand cDNA was synthesized with fragmented mRNA as template and random hexamers as primers, followed by second-strand synthesis with addition of PCR buffer, dNTPs, RNase H and DNA polymerase I. Purification of cDNA was processed with AMPure XP beads.
(4) Double-strand cDNA was subjected to end repair. Adenosine was added to the end and ligated to adapters. AMPure XP beads were applied here to select fragments within size range of 300-400 bp.
(5) cDNA library was obtained by certain rounds of PCR on cDNA fragments generated from step 4.

### Library Quality Control

In order to ensure the quality of library, Qubit 2.0 and Agilent 2100 were used to examine the concentration of cDNA and insert size. Q-PCR was processed to obtain a more accurate library concentration. Library with concentration larger than 2 nM is acceptable.

### Sequencing

The qualified library was sequenced by the high-throughput sequencing platform with PE150 mode. Clean data with high quality was obtained by filtering Raw data, which removes adapter sequence and reads with low quality. These clean data were further mapped to pre-defined reference genome generating mapped data. Assessment on insert size and sequencing randomness were processed on mapped data as library quality control. Basic analysis on mapped data included gene expression quantification, alternative splicing analysis, novel genes prediction and genes structure optimization.

### Sequencing Quality Control

To ensure the quality of the reads before moving onto analysis, raw data containing data such as primers, adapters, etc., were removed before analysis. Procedures for data quality control were listed as follow:
(1) Trim adapter contaminations
(2) Remove nucleotides with low Quality-score.

### Data Alignment to Reference Genome

The reference genome was pre-defined for the analysis. Version information of the reference genome is: Zea_mays.Zm_B73_REFERENCE_NAM_5.0.genome.fa.

### Novel Gene Discovery

In order to optimize the annotation information of a genome, discovery of novel transcripts and genes were achieved by StringTie on base of reference genome. The mapped reads were assembled and compared with original annotations of the genome. The transcript regions without annotation obtained by above processes are defined as novel transcripts. Excluding short transcripts (coding peptides with less than 50 amino acids) or those containing only one exons, 23,552 novel genes were discovered in this study.

### Functional Annotation of Novel Genes

Novel genes were annotated by DIAMOND against databases including NR, Swiss-Prot, COG, KOG and KEGG. KEGG Orthology of novel genes were obtained by above processes. GO Orthology of novel genes were obtained by the underlying software InterProScan basic on the InterPro database. The amino acid sequences of novel genes were blasted against Pfam database by HMMER to gain the annotation information.

### Differential Expression Analysis

The expression of a gene can be influenced by both external stimuli and internal environment, which is highly temporal-specific and tissue-specific. The genes expressed significantly different under different conditions, such as treatment vs control, wild type vs mutants, different time points, different tissue, etc., are defined as Differentially Expressed Genes (DEG). Similarly, transcripts with significantly different expression level are named Differentially Expressed Transcript (DET). The collection of genes acquired in differential expression analysis is defined as DEG set. In result files, the gene sets were named as “A_VS_B” to specify the comparing pair. Normally, “A” represents control group, wild type or former time point. “B” normally represents corresponding treated group, mutant or later time point. The genes with a higher expression level in B than A are defined as up-regulated genes.

The ones with lower expression level in B are defined as down-regulated genes. Therefore, up-reg and down-reg are relative definitions, which relies on the order of A and B.

Criteria for differentially expressed genes were set as Fold Change (FC)≥1.5 and FDR<0.01.

Fold change (FC) refers to the ratio of gene expression in two samples. False Discovery Rate (FDR) refers to adjusted p-value, which is used to measure significancy of difference.

### GO Analysis on DEGs

GO (Gene Ontology) database is a structured biological annotation system established in 2000 containing a standard vocabulary of gene and gene products functions. GO annotation system is a directed acyclic graph containing three main branches: Biological Process, Molecular Function and Cellular Component.

### GO Enrichment Analysis on DEGs

DEGs were then subjected to functional enrichment analysis and the enriched GO terms and corresponding inclusion relationships were shown in the directed acyclic graph.

### KEGG Annotation of Differentially Expressed Genes

In biological organisms, series of gene products are working synergistically to perform biological functions, which is so called pathway. Annotating genes within pathway networks could largely benefit further analysis on biological functions. KEGG (Kyoto Encyclopedia of Genes and Genomes) is one of the major databases on pathways, including metabolic pathways of carbohydrates, nucleotides, amino acids and biological degradation of organics. Besides metabolic pathways, KEGG contains comprehensive description on enzymes involved in the pathways, including amino acid sequences, links to PDB database, etc.

### KEGG Pathway Enrichment Analysis on DEGs

In this study, we examined if the pathways are over-presented with DEGs. Enrichment factors and fisher test were applied in the determination of enrichment degree and significancy of the pathway.

Data obtained from the above studies is shown in Table 4 below.

It is evident from the data that the application of composition disclosed herein increases expression of genes associate with biochemical or physiological pathway is selected from the group consisting of Amino Acid Transport and Metabolism, Anti-Oxidant Activity, Aquaporin Biosynthesis, Auxin Biosynthesis, Sensing and Response, C4 Photosynthesis and Metabolism, Carotenoid Biosynthesis, Chromatin—Epigenetics, Heat Shock Proteins and Transcription Factors, Phenylpropanoid Biosynthesis, Photosynthesis related genes, Transporters, Trehalose, WRKY Transcription Factors, and a combination thereof.

### Example 3: Molecular Gene Expression Associated with DAP-Based Formulation

### Study Design and Horticultural Methods

This study was conducted to evaluate molecular gene expression in shoot and root tissues following foliar application of a DAP-based agrochemical composition comprising Formulation 3. Corn plants (Zea mays, relative maturity 115 days) were grown in plastic nursery pots, each containing approximately 11 liters of substrate. The substrate consisted of a nitrogen- and phosphorus-free Berger's BM2 custom blend. Pots were arranged in a randomized complete block design across greenhouse benches.

The greenhouse compartment was maintained under LED lighting with a 16-hour photoperiod.

Temperature thresholds were set at 23° C. during the day and 28° C. at night for the first week.

During the second week, the thresholds were adjusted to 26° C. during the day and 21° C. at night. From the third week onward, temperatures were maintained at 29° C. during the day and 24° C. at night. Relative humidity was maintained at approximately 65% using water misters.

Plants were irrigated to saturation prior to sowing. Beginning five days after sowing, plants received a nitrogen- and phosphorus-free Hoagland's nutrient solution at 25% strength. At ten days after sowing, the solution concentration was increased to 50% strength. At approximately eighteen days after sowing, ammonium nitrate was added to establish a sufficient nitrogen environment. This design allowed evaluation of formulation-driven molecular responses independent of nutrient deprivation.

At the V6 growth stage, foliar treatments were applied using a fabricated boom sprayer equipped with TeeJet 11002 VS nozzles positioned approximately 18 inches above the plant canopy. Treatments included water, diammonium phosphate (DAP), Formulation 3 applied at 6 L/Ha, and a formulation blank. DAP application rates were adjusted to match the nitrogen and phosphorus content of Formulation 3.

TABLE 3
Treatments, Application Rates, and Sampling Timepoints

| | Application | |
|---|---|---|
| Product | Rate | Timepoints |
| Water | 400 L/Ha | 4, 24, and 96 |
| | | hours after |
| | | application |
| DAP | Rate matched to | 4, 24, and 96 |
| | Formulation 3 | hours after |
| | nutrient input | application |
| Formulation 3 | 6 L/Ha | 4, 24, and 96 |
| | | hours after |
| | | application |
| Formulation 3 | DAP + | 4, 24, and 96 |
| Blank | 6 L/Ha | hours after |
| | | application |

### Sample Collection

Shoot tissue samples were collected from the middle of the youngest mature leaf with the midrib removed. Root samples consisted of seminal root tissue collected from the central region of the root structure. Approximately 0.5×0.5 inch of leaf tissue and approximately 1 gram of root tissue were collected per sample.

All tissue samples were flash-frozen in liquid nitrogen within two minutes of collection and maintained in liquid nitrogen for at least 15 minutes before storage at −80° C. Samples were shipped on dry ice to a transcriptomics laboratory for further analysis.

### Sample Analysis

A total of samples generated high-throughput RNA sequencing data. Paired-end sequencing generated hundreds of gigabytes of clean data, with each sample yielding at least 6 GB of high-quality sequence data. Quality metrics indicated that more than 90% of reads achieved a quality score of Q30 or higher.

### RNA Quality Assessment

RNA purity, concentration, and integrity were evaluated using NanoDrop spectrophotometry, Qubit fluorometric quantification, and Agilent 2100 Bioanalyzer analysis. Only RNA samples meeting quality thresholds for downstream sequencing were advanced to library preparation.

### Library Construction

Qualified RNA was used to construct sequencing libraries according to standard protocols. Messenger RNA was isolated using oligo-dT magnetic beads and fragmented prior to first-strand and second-strand cDNA synthesis. Following end repair and adapter ligation, cDNA fragments in the range of 300-400 bp were selected using AMPure XP beads. Libraries were amplified through PCR to generate sufficient material for sequencing.

### Library Quality Control

Library concentration and insert size were evaluated using Qubit 2.0 fluorometry and Agilent 2100 Bioanalyzer analysis. Quantitative PCR was used to obtain accurate library concentrations. Libraries exceeding 2 nM were deemed acceptable for sequencing.

### Sequencing

Libraries were sequenced using a high-throughput sequencing platform in paired-end 150 bp (PE150) mode. Raw data were processed to remove adapter sequences and low-quality reads, generating high-quality clean data sets for downstream analysis.

### Sequencing Quality Control

Sequencing data were evaluated for insert size distribution, sequencing randomness, and base-quality score distribution. Adapter contaminants and low-quality nucleotides were removed prior to analysis to ensure high confidence in mapped reads.

### Data Alignment to Reference Genome

Clean reads were aligned to the Zea mays reference genome (Zm_B73_REFERENCE_NAM_5.0.genome.fa). Mapping efficiencies ranged from approximately 79% to 93%. Alignment data were used for gene expression quantification, transcript assembly, and alternative splicing analysis.

### Novel Gene Discovery

Mapped reads were assembled using StringTie to identify transcripts not present in the reference genome annotation. Excluding short or single-exon transcripts, more than twenty thousand novel genes were identified in this study.

### Functional Annotation of Novel Genes

Novel genes were functionally annotated using DIAMOND searches against NR, Swiss-Prot, COG, KOG, and KEGG databases. Gene ontology annotations were obtained using InterProScan, and domain annotations were identified through Pfam analysis.

### Differential Expression Analysis

Differentially expressed genes (DEGs) were identified by comparing treated and control samples using criteria of fold change≥1.5 and false discovery rate (FDR)<0.01. Genes with higher expression in treated samples relative to controls were classified as up-regulated, while genes with decreased expression were classified as down-regulated.

### GO Analysis on DEGs

Identified DEGs were assigned gene ontology terms across biological process, molecular function, and cellular component categories to characterize functional responses to the DAP-based formulation.

### GO Enrichment Analysis on DEGs

Enrichment analysis was performed to identify over-represented GO terms among DEGs. Directed acyclic graph representations were used to illustrate relationships among enriched biological processes.

### KEGG Annotation of Differentially Expressed Genes

Differentially expressed genes were annotated against the KEGG database to identify metabolic and signaling pathways affected by application of Formulation 3.

### KEGG Pathway Enrichment Analysis on DEGs

KEGG pathway enrichment analysis revealed that application of the DAP-based Formulation 3 increased expression of genes associated with pathways selected from the group consisting of Amino Acid Transport and Metabolism, Anti-Oxidant Activity, Aquaporin Biosynthesis, Auxin Biosynthesis, Sensing and Response, C4 Photosynthesis and Metabolism, Carotenoid Biosynthesis, Chromatin—Epigenetics, Heat Shock Proteins and Transcription Factors, Phenylpropanoid Biosynthesis, Photosynthesis-Related Genes, Transporters, Trehalose Metabolism, WRKY Transcription Factors, and combinations thereof.

These data demonstrate that Formulation 3 induces a coordinated, temporally staged differential gene expression profile supporting enhanced nutrient assimilation, resource translocation, stress resilience, and growth-associated physiological processes. Data obtained from the above studies is shown in Table 4 below.

TABLE 4

| Biochemistry/ | Sample | |
|---|---|---|
| Physiology | Set | Kegg Annotations of Upregulated Genes |
| Carotenoid | Urea | (+)-abscisic acid 8′-hydroxylase [EC: 1.14.14.137]; putative cytochrome P450 |
| Biosynthesis | | superfamily protein precursor. |
| Carotenoid | Formulation 2 | abscisic-aldehyde oxidase [EC: 1.2.3.14]; indole-3-acetaldehyde oxidase, |
| Biosynthesis | | zeaxanthin epoxidase [EC: 1.14.15.21]; zeaxanthin epoxidase, (+)-abscisic acid |
| | | 8′-hydroxylase [EC: 1.14.14.137]; umc1299; uncharacterized protein |
| | | LOC100274595 precursor, 9-cis-epoxycarotenoid dioxygenase |
| | | [EC: 1.13.11.51]; uncharacterized protein LOC100501454, 9-cis- |
| | | epoxycarotenoid dioxygenase [EC: 1.13.11.51]; uncharacterized protein |
| | | LOC100281391, 15-cis-phytoene desaturase [EC: 1.3.5.5]; phytoene |
| | | dehydrogenase, chloroplastic/chromoplastic, violaxanthin de-epoxidase |
| | | [EC: 1.23.5.1]; violaxanthin de-epoxidase. |
| Carotenoid | Formulation 1 | 9-cis-epoxycarotenoid dioxygenase [EC: 1.13.11.51]; uncharacterized protein |
| Biosynthesis | | LOC100281391. |
| Carotenoid | Formulation 3 | zeaxanthin epoxidase; 9-cis-epoxycarotenoid dioxygenase (NCED); (+)-abscisic |
| Biosynthesis | | acid 8′-hydroxylase |
| Anti-Oxidant | Urea | catalase [EC: 1.11.1.6]; cat2; catalase isozyme 2, L-ascorbate peroxidase |
| Activity | | [EC: 1.11.1.11]; Thylakoid lumenal 29 kDa protein chloroplastic precursor, |
| | | cytokinin dehydrogenase [EC: 1.5.99.12]; CKX10; cytokinin dehydrogenase 10, |
| | | soluble epoxide hydrolase/lipid-phosphate phosphatase [EC: 3.3.2.10 |
| | | 3.1.3.76]; uncharacterized protein LOC100841009, soluble epoxide hydrolase/ |
| | | lipid-phosphate phosphatase [EC: 3.3.2.10 3.1.3.76]; uncharacterized protein |
| | | LOC8071538, L-ascorbate peroxidase [EC: 1.11.1.11]; L-ascorbate peroxidase 1, |
| | | cytosolic. |
| Anti-Oxidant | Formulation 2 | peroxisomal membrane protein 2; Peroxisomal membrane 22 kDa |
| Activity | | (Mpv17/PMP22) family protein precursor, soluble epoxide hydrolase/lipid- |
| | | phosphate phosphatase [EC: 3.3.2.10 3.1.3.76]; LOW QUALITY PROTEIN: |
| | | uncharacterized protein LOC111302718, glutathione S-transferase |
| | | [EC: 2.5.1.18]; IDP340; uncharacterized protein LOC100216831, abscisic- |
| | | aldehyde oxidase [EC: 1.2.3.14]; indole-3-acetaldehyde oxidase, glutathione S- |
| | | transferase [EC: 2.5.1.18]; gst1; glutathione S-transferase 1, zeaxanthin |
| | | epoxidase [EC: 1.14.15.21]; zeaxanthin epoxidase, (+)-abscisic acid 8′- |
| | | hydroxylase [EC: 1.14.14.137]; umc1299; uncharacterized protein |
| | | LOC100274595 precursor, UDP-glucosyltransferase 85A [EC: 2.4.1.-]; UDP- |
| | | glycosyltransferase 85A2 isoform X2, 9-cis-epoxycarotenoid dioxygenase |
| | | [EC: 1.13.11.51]; uncharacterized protein LOC100501454, glutathione S- |
| | | transferase [EC: 2.5.1.18]; gst42; glutathione S-transferase42, 9-cis- |
| | | epoxycarotenoid dioxygenase [EC: 1.13.11.51]; uncharacterized protein |
| | | LOC100281391, peroxin-5; uncharacterized protein LOC100191477, |
| | | spermidine synthase [EC: 2.5.1.16]; spesyn; spermine synthase1, cytokinin trans- |
| | | hydroxylase; cytokinin hydroxylase, UDP-glucosyltransferase 85A [EC: 2.4.1.-]; |
| | | UDP-glycosyltransferase 85A5 isoform X1, 15-cis-phytoene desaturase |
| | | [EC: 1.3.5.5]; phytoene dehydrogenase, chloroplastic/chromoplastic, |
| | | violaxanthin de-epoxidase [EC: 1.23.5.1]; violaxanthin de-epoxidase, pyridoxine |
| | | 4-dehydrogenase [EC: 1.1.1.65]; predicted protein, cytokinin dehydrogenase |
| | | [EC: 1.5.99.12]; cko3; cytokinin oxidase 3 precursor, pyridoxine 4- |
| | | dehydrogenase [EC: 1.1.1.65]; uncharacterized protein LOC100193172. |
| Anti-Oxidant | Formulation 1 | L-ascorbate peroxidase [EC: 1.11.1.11]; L-ascorbate peroxidase 1, cytosolic, |
| Activity | | glutathione S-transferase [EC: 2.5.1.18]; IDP340; uncharacterized protein |
| | | LOC100216831, UDP-glucosyltransferase 85A [EC: 2.4.1.-]; UDP- |
| | | glycosyltransferase 85A2 isoform X2, 9-cis-epoxycarotenoid dioxygenase |
| | | [EC: 1.13.11.51]; uncharacterized protein LOC100281391, glutathione S- |
| | | transferase [EC: 2.5.1.18]; csu454(gst); uncharacterized protein LOC100282447, |
| | | glutathione S-transferase [EC: 2.5.1.18]; GST39; glutathione transferase39, |
| | | monodehydroascorbate reductase (NADH) [EC: 1.6.5.4]; monodehydroascorbate |
| | | reductase 4, peroxisomal-like, glucuronokinase [EC: 2.7.1.43]; LOC109763399; |
| | | probable glucuronokinase 2, ribonucleoside-diphosphate reductase subunit M2 |
| | | [EC: 1.17.4.1]; uncharacterized protein LOC100192012, glutathione S- |
| | | transferase [EC: 2.5.1.18]; IN2-1 protein, aldehyde dehydrogenase (NAD+) |
| | | [EC: 1.2.1.3]; aldehyde dehydrogenase 5, mitochondrial-like, sarcosine oxidase/ |
| | | L-pipecolate oxidase [EC: 1.5.3.1 1.5.3.7]; uncharacterized protein |
| | | LOC100283462, (S)-2-hydroxy-acid oxidase [EC: 1.1.3.15]; go1; glycolate |
| | | oxidase 1. |
| Anti-Oxidant | Formulation 3 | glutathione S-transferases (gst1, gst42); glutaredoxin 3; thioredoxin 1 |
| Activity | | |
| Amino Acid | Urea | solute carrier family 15 (peptide/histidine transporter), member 3/4; peptide |
| Transport and | | transporter PTR2, indole-3-glycerol-phosphate lyase [EC: 4.1.2.8]; indole-3- |
| Metabolism | | glycerol phosphate lyase, chloroplastic-like, basic amino acid/polyamine |
| | | antiporter, APA family; IDP630; Cationic amino acid transporter 1, vacuolar |
| | | protein sorting-associated protein IST1; hypothetical protein, aminoacylase |
| | | [EC: 3.5.1.14]; aminoacylase-1, glutamine-fructose-6-phosphate transaminase |
| | | (isomerizing) [EC: 2.6.1.16]; uncharacterized protein LOC100194010, 3- |
| | | methylcrotonyl-CoA carboxylase alpha subunit [EC: 6.4.1.4]; methylcrotonoyl- |
| | | CoA carboxylase subunit alpha, mitochondrial. |
| Amino Acid | Formulation 2 | solute carrier family 15 (peptide/histidine transporter), member 3/4; pco137042; |
| Transport and | | uncharacterized protein LOC100281589, indole-3-glycerol phosphate synthase |
| Metabolism | | [EC: 4.1.1.48]; indole-3-glycerol phosphate synthase, 5- |
| | | methyltetrahydropteroyltriglutamatehomocysteine methyltransferase |
| | | [EC: 2.1.1.14]; csu503(met); uncharacterized LOC541942, delta-1-pyrroline-5- |
| | | carboxylate synthetase [EC: 2.7.2.11 1.2.1.41]; AY109740; uncharacterized |
| | | LOC778431, arogenate/prephenate dehydratase [EC: 4.2.1.91 |
| | | 4.2.1.51]; uncharacterized protein LOC100193934, solute carrier family 32 |
| | | (vesicular inhibitory amino acid transporter); amino acid transporter AVT1I, |
| | | spermidine synthase [EC: 2.5.1.16]; spesyn; spermine synthase1, choline |
| | | transport protein; choline transport protein-like, glutamine synthetase |
| | | [EC: 6.3.1.2]; gln3; glutamine synthetase root isozyme 5, thiosulfate/3- |
| | | mercaptopyruvate sulfurtransferase [EC: 2.8.1.1 2.8.1.2]; thiosulfate/3- |
| | | mercaptopyruvate sulfurtransferase 2 isoform X2, malate dehydrogenase |
| | | [EC: 1.1.1.37]; malate dehydrogenase, cytoplasmic-like, formamidase |
| | | [EC: 3.5.1.49]; uncharacterized protein LOC100274074, polyamine oxidase |
| | | [EC: 1.5.3.17 1.5.3.-]; probable polyamine oxidase 2, |
| | | aminocyclopropanecarboxylate oxidase [EC: 1.14.17.4]; ACO31; acc oxidase, |
| | | isovaleryl-CoA dehydrogenase [EC: 1.3.8.4]; uncharacterized protein |
| | | LOC100382372. |
| Amino Acid | Formulation 1 | solute carrier family 32 (vesicular inhibitory amino acid transporter); amino acid |
| Transport and | | transporter AVT1I, glutamate decarboxylase [EC: 4.1.1.15]; glutamate |
| Metabolism | | decarboxylase 2, vacuolar protein sorting-associated protein IST1; hypothetical |
| | | protein, glucuronokinase [EC: 2.7.1.43]; LOC109763399; probable |
| | | glucuronokinase 2, aminoacylase [EC: 3.5.1.14]; aminoacylase-1, 2- |
| | | oxoisovalerate dehydrogenase E1 component alpha subunit |
| | | [EC: 1.2.4.4]; pco079304; uncharacterized protein LOC100191513, 6- |
| | | phosphofructokinase 1 [EC: 2.7.1.11]; ATP-dependent 6-phosphofructokinase 6- |
| | | like, malate dehydrogenase [EC: 1.1.1.37]; malate dehydrogenase, cytoplasmic- |
| | | like, sarcosine oxidase/L-pipecolate oxidase [EC: 1.5.3.1 |
| | | 1.5.3.7]; uncharacterized protein LOC100283462, aldehyde dehydrogenase |
| | | (NAD+) [EC: 1.2.1.3]; aldehyde dehydrogenase 5, mitochondrial-like. |
| Amino Acid | Formulation 3 | glutamine synthetase (gln3; root isozyme 5); aminotransferases; amino acid |
| Transport and | | transporters (AVT1I); spermidine synthase |
| Metabolism | | |
| Photosynthesis | Urea | sucrose synthase [EC: 2.4.1.13]; sus2; sucrose synthase 2, trehalose 6-phosphate |
| related genes | | synthase/phosphatase [EC: 2.4.1.15 3.1.3.12]; gpm600; uncharacterized protein |
| | | LOC100281927, trehalose 6-phosphate synthase/phosphatase [EC: 2.4.1.15 |
| | | 3.1.3.12]; alpha,alpha-trehalose-phosphate synthase [UDP-forming] 6, trehalose |
| | | 6-phosphate synthase/phosphatase [EC: 2.4.1.15 3.1.3.12]; uncharacterized |
| | | protein LOC103654185, malate dehydrogenase (oxaloacetate- |
| | | decarboxylating)(NADP+) [EC: 1.1.1.40]; me4; NADP malic enzyme 4, |
| | | chaperonin GroEL; ruBisCO large subunit-binding protein subunit beta, |
| | | chloroplastic, light-harvesting complex II chlorophyll a/b binding protein |
| | | 1; chlorophyll a-b binding protein 2, light-harvesting complex I chlorophyll a/b |
| | | binding protein 4; uncharacterized protein LOC100280410, light-harvesting |
| | | complex II chlorophyll a/b binding protein 4; uncharacterized protein |
| | | LOC100216729, photosystem II Psb27 protein; photosystem II 11 kD protein, |
| | | catalase [EC: 1.11.1.6]; cat2; catalase isozyme 2, alpha-amylase |
| | | [EC: 3.2.1.1]; alpha-amylase isozyme 2A, glucan endo-1,3-beta-glucosidase 4 |
| | | [EC: 3.2.1.39]; glucan endo-1,3-beta-glucosidase 4-like isoform X2, beta- |
| | | glucosidase [EC: 3.2.1.21]; beta-glucosidase 12, malate synthase |
| | | [EC: 2.3.3.9]; mas1; malate synthase, glyoxysomal, beta-amylase |
| | | [EC: 3.2.1.2]; pco104637; beta-amylase, glucan endo-1,3-beta-glucosidase 1/2/3 |
| | | [EC: 3.2.1.39]; hypothetical protein, inositol 3-alpha-galactosyltransferase |
| | | [EC: 2.4.1.123]; galactinol synthase 1. |
| Photosynthesis | Formulation 2 | chaperonin GroEL; ruBisCO large subunit-binding protein subunit beta, |
| related genes | | chloroplastic-like, chaperonin GroEL; uncharacterized protein LOC100272259, |
| | | trehalose 6-phosphate synthase/phosphatase [EC: 2.4.1.15 |
| | | 3.1.3.12]; uncharacterized protein LOC103654185, malate dehydrogenase |
| | | [EC: 1.1.1.37]; malate dehydrogenase, cytoplasmic-like, trehalose 6-phosphate |
| | | phosphatase [EC: 3.1.3.12]; trehalose-phosphate phosphatase A-like, chaperonin |
| | | GroEL; ruBisCO large subunit-binding protein subunit beta, chloroplastic, |
| | | trehalose 6-phosphate synthase/phosphatase [EC: 2.4.1.15 3.1.3.12]; probable |
| | | alpha,alpha-trehalose-phosphate synthase [UDP-forming] 9, trehalose 6- |
| | | phosphate synthase/phosphatase [EC: 2.4.1.15 3.1.3.12]; probable alpha,alpha- |
| | | trehalose-phosphate synthase [UDP-forming] 10, trehalose 6-phosphate |
| | | synthase/phosphatase [EC: 2.4.1.15 3.1.3.12]; alpha,alpha-trehalose-phosphate |
| | | synthase [UDP-forming] 1, trehalose 6-phosphate synthase/phosphatase |
| | | [EC: 2.4.1.15 3.1.3.12]; trehalose-6-phosphate synthase, ferredoxin; fdx2; |
| | | ferredoxin-2, chloroplastic, UDP-glucosyltransferase 85A [EC: 2.4.1.-]; UDP- |
| | | glycosyltransferase 85A2 isoform X2, UDP-glucosyltransferase 85A |
| | | [EC: 2.4.1.-]; UDP-glycosyltransferase 85A5 isoform X1, fumarate hydratase, |
| | | class II [EC: 4.2.1.2]; pco106335; uncharacterized protein LOC100279871, |
| | | glutamine synthetase [EC: 6.3.1.2]; gln3; glutamine synthetase root isozyme 5, |
| | | hexokinase [EC: 2.7.1.1]; hexokinase-3, glycogen phosphorylase |
| | | [EC: 2.4.1.1]; pho1; alpha-1,4-glucan phosphorylase, geranylgeranyl |
| | | diphosphate/geranylgeranyl-bacteriochlorophyllide a reductase [EC: 1.3.1.83 |
| | | 1.3.1.111]; geranylgeranyl diphosphate reductase, chloroplastic, light-harvesting |
| | | complex II chlorophyll a/b binding protein 1; chlorophyll a-b binding protein 2, |
| | | light-harvesting complex I chlorophyll a/b binding protein 4; uncharacterized |
| | | protein LOC100280410, light-harvesting complex II chlorophyll a/b binding |
| | | protein 4; uncharacterized protein LOC100216729, photosystem II Psb27 |
| | | protein; photosystem II 11 kD protein, alpha-amylase [EC: 3.2.1.1]; alpha- |
| | | amylase isozyme 2A, glucan endo-1,3-beta-glucosidase 4 [EC: 3.2.1.39]; glucan |
| | | endo-1,3-beta-glucosidase 4-like isoform X2, sucrose synthase |
| | | [EC: 2.4.1.13]; sus2; sucrose synthase 2, beta-amylase [EC: 3.2.1.2]; pco104637; |
| | | beta-amylase, trehalose 6-phosphate synthase/phosphatase [EC: 2.4.1.15 |
| | | 3.1.3.12]; gpm600; uncharacterized protein LOC100281927, trehalose 6- |
| | | phosphate synthase/phosphatase [EC: 2.4.1.15 3.1.3.12]; alpha,alpha-trehalose- |
| | | phosphate synthase [UDP-forming] 6, glucan endo-1,3-beta-glucosidase 1/2/3 |
| | | [EC: 3.2.1.39]; hypothetical protein, formamidase [EC: 3.5.1.49]; uncharacterized |
| | | protein LOC100274074, alpha-galactosidase [EC: 3.2.1.22]; uncharacterized |
| | | protein LOC100280970 precursor, alpha-galactosidase |
| | | [EC: 3.2.1.22]; uncharacterized protein LOC100191764 precursor. |
| Photosynthesis | Formulation 1 | trehalose 6-phosphate phosphatase [EC: 3.1.3.12]; probable trehalose-phosphate |
| related genes | | phosphatase 7, sucrose synthase [EC: 2.4.1.13]; sucrose synthase 7, malate |
| | | dehydrogenase [EC: 1.1.1.37]; malate dehydrogenase, cytoplasmic-like, 6- |
| | | phosphofructokinase 1 [EC: 2.7.1.11]; ATP-dependent 6-phosphofructokinase 6- |
| | | like, light-harvesting complex II chlorophyll a/b binding protein 1; chlorophyll |
| | | a-b binding protein 2, light-harvesting complex II chlorophyll a/b binding |
| | | protein 5; Lhcb5-2; chlorophyll a/b-binding apoprotein CP26 precursor, |
| | | ferredoxin; fdx5; ferredoxin-5, chloroplastic, ferredoxin; fdx2; ferredoxin-2, |
| | | chloroplastic, UDP-glucosyltransferase 85A [EC: 2.4.1.-]; UDP- |
| | | glycosyltransferase 85A2 isoform X2, glutamate receptor, ionotropic, |
| | | plant; glutamate receptor 3.1, glucan 1,3-beta-glucosidase [EC: 3.2.1.58]; glucan |
| | | 1,3-beta-glucosidase-like, alpha-amylase [EC: 3.2.1.1]; alpha-amylase isozyme |
| | | 2A, glucan endo-1,3-beta-glucosidase 1/2/3 [EC: 3.2.1.39]; glucan endo-1,3- |
| | | beta-glucosidase 3-like, glycogenin [EC: 2.4.1.186]; glycosyl transferase, |
| | | families GT8 + GT64, granule-bound starch synthase [EC: 2.4.1.242]; wx1; |
| | | granule-bound starch synthase 1, chloroplastic/amyloplastic precursor, beta- |
| | | galactosidase [EC: 3.2.1.23]; beta-galactosidase 2, raffinose synthase |
| | | [EC: 2.4.1.82]; probable galactinolsucrose galactosyltransferase 2, aldehyde |
| | | dehydrogenase (NAD+) [EC: 1.2.1.3]; aldehyde dehydrogenase 5, mitochondrial- |
| | | like, UDP-glucose 4-epimerase [EC: 5.1.3.2]; UDP-glucose-4-epimerase, |
| | | ribulose-bisphosphate carboxylase small chain [EC: 4.1.1.39]; ribulose |
| | | bisphosphate carboxylase small subunit 2, (S)-2-hydroxy-acid oxidase |
| | | [EC: 1.1.3.15]; go1; glycolate oxidase 1. |
| Photosynthesis | Formulation 3 | magnesium chelatase subunit D (ChlD); magnesium chelatase subunit I (oy1; |
| related genes | | oil yellow 1); photosystem II PsbW protein; photosystem II oxygen-evolving |
| | | enhancer protein 1 (OEE1); photosystem II oxygen-evolving enhancer protein 3 |
| | | (OEE3); photosystem I reaction center subunits II, III, VI, X (psaH1, psa6); |
| | | light-harvesting complex proteins (lhcb5, chlorophyll a/b-binding |
| | | proteins 1, 3, 5*, LOC identifiers provided*) |
| Phenylpropanoid | Urea | beta-glucosidase [EC: 3.2.1.21]; beta-glucosidase 12, peroxidase [EC: 1.11.1.7]; |
| Biosynthesis | | gpm233; uncharacterized protein LOC100273479 precursor. |
| Phenylpropanoid | Formulation 2 | caffeoyl-CoA O-methyltransferase [EC: 2.1.1.104]; uncharacterized protein |
| Biosynthesis | | LOC107282093, peroxidase [EC: 1.11.1.7]; putative Peroxidase 48, peroxidase |
| | | [EC: 1.11.1.7]; ap1; anionic peroxidase precursor, peroxidase [EC: 1.11.1.7]; |
| | | peroxidase 47, peroxidase [EC: 1.11.1.7]; uncharacterized protein |
| | | LOC100216644, peroxidase [EC: 1.11.1.7]; peroxidase 50. |
| Phenylpropanoid | Formulation 1 | caffeoyl-CoA O-methyltransferase [EC: 2.1.1.104]; uncharacterized protein |
| Biosynthesis | | LOC107282093, cinnamyl-alcohol dehydrogenase [EC: 1.1.1.195]; cl4921_1; |
| | | uncharacterized protein LOC100281336, peroxidase [EC: 1.11.1.7]; putative |
| | | Peroxidase 48, shikimate O-hydroxycinnamoyltransferase [EC: 2.3.1.133]; |
| | | putrescine hydroxycinnamoyltransferase 1 isoform X1, peroxidase |
| | | [EC: 1.11.1.7]; umc2588; uncharacterized protein LOC100286338 precursor, |
| | | peroxidase [EC: 1.11.1.7]; pco073925; peroxidase 42 precursor, cinnamoyl-CoA |
| | | reductase [EC: 1.2.1.44]; LOC109780731; cinnamoyl-CoA reductase 1-like, |
| | | peroxidase [EC: 1.11.1.7]; peroxidase 44, 4-coumarateCoA ligase [EC: 6.2.1.12]; |
| | | pco063262; uncharacterized protein LOC542166. |
| Transporters | Urea | catalase [EC: 1.11.1.6]; cat2; catalase isozyme 2, solute carrier family 15 |
| | | (peptide/histidine transporter), member 3/4; peptide transporter PTR2, ATP- |
| | | binding cassette, subfamily C (CFTR/MRP), member 1 [EC: 7.6.2.3]; ABC |
| | | transporter C family member 3, ATP-binding cassette, subfamily A (ABC1), |
| | | member 3; SmABCA1; ATP-binding cassette transporter, vacuolar protein |
| | | sorting-associated protein IST1; hypothetical protein, MFS transporter, PHS |
| | | family, inorganic phosphate transporter; pht2; phosphate transporter protein 2, |
| | | basic amino acid/polyamine antiporter, APA family; IDP630; Cationic amino |
| | | acid transporter 1. |
| Transporters | Formulation 2 | protein transport protein SEC61 subunit alpha; uncharacterized protein |
| | | LOC100217058 isoform X1, magnesium transporter; uncharacterized protein |
| | | LOC100501448 isoform X1, solute carrier family 35, member E3; similar to |
| | | glucose-6-phosphate/phosphate translocator, thiosulfate/3-mercaptopyruvate |
| | | sulfurtransferase [EC: 2.8.1.1 2.8.1.2]; thiosulfate/3-mercaptopyruvate |
| | | sulfurtransferase 2 isoform X2, MFS transporter, SP family, sugar: H+ |
| | | symporter; hexose carrier protein HEX6, 5- |
| | | methyltetrahydropteroyltriglutamatehomocysteine methyltransferase |
| | | [EC: 2.1.1.14]; csu503(met); uncharacterized LOC541942, solute carrier family |
| | | 25 (mitochondrial phosphate transporter), member 23/24/25/41; adenine |
| | | nucleotide transporter BT1, chloroplastic/mitochondrial-like, MFS transporter, |
| | | SP family, solute carrier family 2 (myo-inositol transporter), member 13; |
| | | probable inositol transporter 2, metal transporter CNNM; cl9255_1; |
| | | uncharacterized protein LOC100283152, KUP system potassium uptake |
| | | protein; probable potassium transporter 4, choline transport protein; choline |
| | | transport protein-like, solute carrier family 15 (peptide/histidine transporter), |
| | | member 3/4; pco137042; uncharacterized protein LOC100281589, P-type Ca2+ |
| | | transporter type 2C [EC: 7.2.2.10]; calcium-transporting ATPase 5, plasma |
| | | membrane-type isoform X1, integrin-linked kinase-associated serine/threonine |
| | | phosphatase 2C [EC: 3.1.3.16]; WAT1-related protein At3g18200 isoform X1, |
| | | MFS transporter, PHS family, inorganic phosphate transporter; inorganic |
| | | phosphate transporter 1-2-like, ATP-binding cassette, subfamily G (WHITE), |
| | | member 2, SNQ2; ABC transporter, ATP-binding protein, ATP-binding |
| | | cassette, subfamily A (ABC1), member 3; ABC transporter A family member 1 |
| | | isoform X1, solute carrier family 32 (vesicular inhibitory amino acid |
| | | transporter); amino acid transporter AVT1I. |
| Transporters | Formulation 1 | glucuronokinase [EC: 2.7.1.43]; LOC109763399; probable glucuronokinase 2, |
| | | metal transporter CNNM; cl9255_1; uncharacterized protein LOC100283152, |
| | | integrin-linked kinase-associated serine/threonine phosphatase 2C |
| | | [EC: 3.1.3.16]; WAT1-related protein At3g18200 isoform X1, ATP-binding |
| | | cassette, subfamily B (MDR/TAP), member 1 [EC: 7.6.2.2]; uncharacterized |
| | | protein LOC111781709, P-type Ca2+ transporter type 2C [EC: 7.2.2.10]; |
| | | probable calcium-transporting ATPase 6, plasma membrane-type isoform X1, |
| | | solute carrier family 32 (vesicular inhibitory amino acid transporter); amino |
| | | acid transporter AVT1I, ATP-binding cassette, subfamily A (ABC1), member |
| | | 3; ABC transporter A family member 1 isoform X1, MFS transporter, PHS |
| | | family, inorganic phosphate transporter; pht1; phosphate transporter protein 1, |
| | | ATP-binding cassette, subfamily G (WHITE), member 2; ABC transporter G |
| | | family member 9-like, ATP-binding cassette, subfamily C (CFTR/MRP), |
| | | member 1 [EC: 7.6.2.3]; ABC transporter C family member 3, vacuolar protein |
| | | sorting-associated protein IST1; hypothetical protein, solute carrier family 45, |
| | | member 1/2/4; umc2347; sucrose transporter 1. |
| Transporters | Formulation 3 | ABC transporters (ATP-binding cassette transport proteins); phosphate |
| | | transporter (PHT family); solute carrier family transporters; nitrate/nitrite |
| | | transporters; amino acid transporters (AVT family) |
| Auxin | Urea | protein brassinosteroid insensitive 1 [EC: 2.7.10.1 2.7.11.1]; brassinosteroid |
| Biosynthesis, | | LRR receptor kinase-like, auxin response factor; pco123854; uncharacterized |
| Sensing and | | protein LOC100384045, calcium-dependent protein kinase [EC: 2.7.11.1]; |
| Response | | IDP294; uncharacterized protein LOC100273840, somatic embryogenesis |
| | | receptor kinase 1 [EC: 2.7.10.1 2.7.11.1]; somatic embryogenesis receptor-like |
| | | kinase 3 precursor, auxin-responsive protein IAA; uncharacterized protein |
| | | LOC100384587, protein brassinosteroid insensitive 1 [EC: 2.7.10.1 2.7.11.1]; |
| | | systemin receptor SR160, cyclin-dependent kinase 12/13 [EC: 2.7.11.22 |
| | | 2.7.11.23]; probable serine/threonine-protein kinase At1g54610, LRR receptor- |
| | | like serine/threonine-protein kinase FLS2 [EC: 2.7.11.1]; LRR receptor-like |
| | | serine/threonine-protein kinase FLS2, kinase [EC: 2.7.1.-]; probable |
| | | serine/threonine-protein kinase PBL19, kinase [EC: 2.7.1.-]; PBL1; PBS1-like 1, |
| | | endo-1,3(4)-beta-glucanase [EC: 3.2.1.6]; uncharacterized protein |
| | | LOC111487562, kinase [EC: 2.7.1.-]; probable receptor-like protein kinase |
| | | At3g55450 isoform X1, serine incorporator 1/3; serine incorporator 3, |
| | | brassinosteroid insensitive 1-associated receptor kinase 1 [EC: 2.7.10.1 |
| | | 2.7.11.1]; BRASSINOSTEROID INSENSITIVE 1-associated receptor kinase 1 |
| | | isoform X1, protein brassinosteroid insensitive 1 [EC: 2.7.10.1 2.7.11.1]; |
| | | systemin receptor SR160-like, EIX receptor 1/2; leucine-rich repeat receptor |
| | | protein kinase EMS1-like, GPN-loop GTPase; LOW QUALITY PROTEIN: |
| | | uncharacterized protein LOC104799061, alpha-amylase [EC: 3.2.1.1]; alpha- |
| | | amylase isozyme 2A, kinase [EC: 2.7.1.-]; receptor protein kinase TMK1-like |
| Auxin | Formulation 2 | mitogen-activated protein kinase kinase kinase ANP1 [EC: 2.7.11.25]; mitogen- |
| Biosynthesis, | | activated protein kinase kinase kinase NPK1-like, coatomer subunit beta′; |
| Sensing and | | LOC109749707; serine/threonine-protein kinase StkP-like isoform X1, auxin |
| Response | | response factor; pco123854; uncharacterized protein LOC100384045, pto- |
| | | interacting protein 1 [EC: 2.7.11.1]; pto-interacting protein 1-like, |
| | | serine/threonine-protein kinase PBS1 [EC: 2.7.11.1]; serine/threonine-protein |
| | | kinase PBS1-like isoform X1, serine/threonine-protein kinase ULK2 |
| | | [EC: 2.7.11.1]; serine/threonine-protein kinase ATG1a, kinase [EC: 2.7.1.-]; |
| | | receptor-like protein kinase HSL1, senescence-induced receptor-like |
| | | serine/threonine-protein kinase; senescence-induced receptor-like |
| | | serine/threonine-protein kinase isoform X2, brassinosteroid insensitive 1- |
| | | associated receptor kinase 1 [EC: 2.7.10.1 2.7.11.1]; BRASSINOSTEROID |
| | | INSENSITIVE 1-associated receptor kinase 1-like isoform X1, kinase |
| | | [EC: 2.7.1.-]; probable serine/threonine-protein kinase PBL19, GPN-loop |
| | | GTPase; LOW QUALITY PROTEIN: uncharacterized protein LOC104799061, |
| | | kinase [EC: 2.7.1.-]; PBL1; PBS1-like 1, auxin-responsive protein IAA; IAA24 - |
| | | auxin-responsive Aux/IAA family member, 5′-AMP-activated protein kinase, |
| | | catalytic alpha subunit [EC: 2.7.11.11]; uncharacterized protein LOC100274837. |
| Auxin | Formulation 1 | mitogen-activated protein kinase kinase kinase ANP1 [EC: 2.7.11.25]; mitogen- |
| Biosynthesis, | | activated protein kinase kinase kinase NPK1-like, coatomer subunit beta′; |
| Sensing and | | LOC109749707; serine/threonine-protein kinase StkP-like isoform X1, auxin |
| Response | | response factor; pco123854; uncharacterized protein LOC100384045, |
| | | aquaporin PIP; pip2e; aquaporin PIP2-1, pto-interacting protein 1 [EC: 2.7.11.1]; |
| | | pto-interacting protein 1-like, aquaporin PIP; pip2f; aquaporin PIP2-6, |
| | | serine/threonine-protein kinase PBS1 [EC: 2.7.11.1]; serine/threonine-protein |
| | | kinase PBS1-like isoform X1, serine/threonine-protein kinase ULK2 |
| | | [EC: 2.7.11.1]; serine/threonine-protein kinase ATG1a, senescence-induced |
| | | receptor-like serine/threonine-protein kinase; senescence-induced receptor-like |
| | | serine/threonine-protein kinase isoform X2, aquaporin TIP; tip1; aquaporin |
| | | TIP1-1, brassinosteroid insensitive 1-associated receptor kinase 1 [EC: 2.7.10.1 |
| | | 2.7.11.1]; BRASSINOSTEROID INSENSITIVE 1-associated receptor kinase |
| | | 1-like isoform X1, kinase [EC: 2.7.1.-]; probable serine/threonine-protein kinase |
| | | PBL19, GPN-loop GTPase; LOW QUALITY PROTEIN: uncharacterized |
| | | protein LOC104799061, kinase [EC: 2.7.1.-]; PBL1; PBS1-like 1, auxin- |
| | | responsive protein IAA; IAA24 - auxin-responsive Aux/IAA family member, |
| | | aquaporin PIP; pip2c; aquaporin PIP2-3, 5′-AMP-activated protein kinase, |
| | | catalytic alpha subunit [EC: 2.7.11.11]; uncharacterized protein LOC100274837. |
| Auxin | Formulation 3 | auxin response factor (ARF); auxin-responsive Aux/IAA protein (IAA24); |
| Biosynthesis, | | brassinosteroid-insensitive 1-associated receptor kinase 1; mitogen-activated |
| Sensing and | | protein kinase kinase kinase (ANP1/NPK1-like); tryptophan biosynthesis genes |
| Response | | |
| WRKY | Urea | probable WRKY transcription factor 52; disease resistance protein RRS1-like. |
| Transcription | | |
| Factors | | |
| WRKY | Formulation 2 | WRKY transcription factor 33; WRKY transcription factor WRKY24-like |
| Transcription | | isoform X1, ATP-dependent RNA helicase DDX42 [EC: 3.6.4.13]; DEAD-box |
| Factors | | ATP-dependent RNA helicase 24 isoform X1, disease resistance protein RPS2; |
| | | disease resistance protein At4g27190, WRKY transcription factor 33; WRKY53 - |
| | | superfamily of TFs having WRKY and zinc finger domains, WRKY |
| | | transcription factor 33; probable WRKY transcription factor 33, WRKY |
| | | transcription factor 1; WRKY transcription factor 1, probable WRKY |
| | | transcription factor 52; disease resistance protein RRS1-like. |
| WRKY | Formulation 1 | hydroxyproline O-galactosyltransferase HPGT [EC: 2.4.1.-]; hydroxyproline O- |
| Transcription | | galactosyltransferase HPGT1, disease resistance protein RPS2; disease |
| Factors | | resistance protein At4g27190, probable WRKY transcription factor 52; disease |
| | | resistance protein RRS1-like, ATP-dependent RNA helicase DDX42 |
| | | [EC: 3.6.4.13]; DEAD-box ATP-dependent RNA helicase 24 isoform X1, |
| | | WRKY transcription factor 1; WRKY transcription factor 1-like, WRKY |
| | | transcription factor 1; WRKY transcription factor 1, WRKY transcription factor |
| | | 33; probable WRKY transcription factor 26, WRKY transcription factor 33; |
| | | probable WRKY transcription factor 33. |
| WRKY | Formulation 3 | WRKY33; WRKY24-like; WRKY1; WRKY52; WRKY26; disease resistance |
| Transcription | | proteins RPS2, RRS1-like |
| Factors | | |
| Heat Shock | Urea | thioredoxin 1; thioredoxin F-type, chloroplastic-like, E3 ubiquitin-protein ligase |
| Proteins and | | ATL4 [EC: 2.3.2.27]; RING-H2 finger protein ATL5, heat shock 70 kDa protein |
| Transcription | | 1/2/6/8; pco083553; heat shock cognate 70 kDa protein 2, molecular chaperone |
| Factors | | DnaJ; chaperone protein DnaJ, E3 ubiquitin-protein ligase ATL10/75/76/77/78 |
| | | [EC: 2.3.2.27]; RING-H2 finger protein ATL74, boron transporter; |
| | | uncharacterized protein LOC4326215, calreticulin; calreticulin-3 precursor, |
| | | calreticulin; gpm494; uncharacterized protein LOC100382232 precursor, |
| | | phytepsin [EC: 3.4.23.40]; aspartic proteinase oryzasin-1, mitochondrial |
| | | chaperone BCS1; AAA-ATPase ASD, mitochondrial-like, aspartyl protease |
| | | family protein [EC: 3.4.23.-]; aspartyl protease family protein 2-like, E3 |
| | | ubiquitin-protein ligase ATL41 [EC: 2.3.2.27]; probable E3 ubiquitin-protein |
| | | ligase ATL44 |
| Heat Shock | Formulation 2 | ATP-dependent Clp protease ATP-binding subunit ClpB; hsp101; heat-shock |
| Proteins and | | protein 101, ATP-dependent Clp protease ATP-binding subunit ClpB; |
| Transcription | | uncharacterized protein LOC100217072 isoform X1, calreticulin; crt2; |
| Factors | | calreticulin precursor, cathepsin D [EC: 3.4.23.5]; aspartic proteinase isoform |
| | | X1, chaperonin GroEL; ruBisCO large subunit-binding protein subunit beta, |
| | | chloroplastic, chaperonin GroEL; ruBisCO large subunit-binding protein |
| | | subunit beta, chloroplastic-like, chaperonin GroEL; uncharacterized protein |
| | | LOC100272259, chaperonin GroES; 10 kDa chaperonin, mitochondrial, |
| | | cytosolic prostaglandin-E synthase [EC: 5.3.99.3]; uncharacterized protein |
| | | OsI_027940-like, DnaJ homolog subfamily A member 2; dnaJ protein homolog |
| | | 2, DnaJ homolog subfamily A member 2; dnaJ protein homolog, DnaJ homolog |
| | | subfamily A member 2; putative dnaJ chaperone family protein, DnaJ homolog |
| | | subfamily A member 5; predicted protein, DnaJ homolog subfamily B member |
| | | 4; dnaJ homolog subfamily B member 1 isoform X1, DnaJ homolog subfamily |
| | | B member 4; dnaJ homolog subfamily B member 4, DnaJ homolog subfamily B |
| | | member 4; dnaJ protein, DnaJ homolog subfamily B member 4; dnaJ subfamily |
| | | B member 5, DnaJ homolog subfamily B member 4; uncharacterized protein |
| | | LOC100382308, E3 ubiquitin-protein ligase ATL10/75/76/77/78 [EC: 2.3.2.27]; |
| | | RING-H2 finger protein ATL78-like, E3 ubiquitin-protein ligase |
| | | ATL6/9/15/31/42/55 [EC: 2.3.2.27]; E3 ubiquitin-protein ligase ATL31, E3 |
| | | ubiquitin-protein ligase ATL6/9/15/31/42/55 [EC: 2.3.2.27]; putative RING zinc |
| | | finger domain superfamily protein precursor, E3 ubiquitin-protein ligase |
| | | RNF115/126 [EC: 2.3.2.27]; E3 ubiquitin-protein ligase RDUF1, E3 ubiquitin- |
| | | protein ligase RNF115/126 [EC: 2.3.2.27]; E3 ubiquitin-protein ligase RING1- |
| | | like, FK506-binding protein 4/5 [EC: 5.2.1.8]; 70 kDa peptidyl-prolyl isomerase, |
| | | FK506-binding protein 4/5 [EC: 5.2.1.8]; peptidyl-prolyl cis-trans isomerase |
| | | FKBP62-like, FK506-binding protein 4/5 [EC: 5.2.1.8]; uncharacterized protein |
| | | LOC100283417, glutaredoxin 3; Grx_C4 - glutaredoxin subgroup I, glutathione |
| | | S-transferase [EC: 2.5.1.18]; gst1; glutathione S-transferase 1, glutathione S- |
| | | transferase [EC: 2.5.1.18]; gst42; glutathione S-transferase42, heat shock 70 kDa |
| | | protein 1/2/6/8; heat shock 70 kDa protein-like, heat shock 70 kDa protein |
| | | 1/2/6/8; heat shock protein 1, heat shock 70 kDa protein 1/2/6/8; probable |
| | | mediator of RNA polymerase II transcription subunit 37c, heat shock 70 kDa |
| | | protein 4; heat shock 70 kDa protein 15, heat shock 70 kDa protein 4; heat shock |
| | | 70 kDa protein 4, heat shock 70 kDa protein 4; heat shock 70 kDa protein 8, heat |
| | | shock protein 90 kDa beta; endoplasmin precursor, heat shock protein 90 kDa |
| | | beta; shpl1; shepherd-like 1 precursor, heat shock transcription factor, other |
| | | eukaryote; cl30385_1; uncharacterized protein LOC100280318, heat shock |
| | | transcription factor, other eukaryote; heat shock factor protein 4, heat shock |
| | | transcription factor, other eukaryote; heat shock factor protein 7, heat shock |
| | | transcription factor, other eukaryote; heat shock factor protein HSF30, heat |
| | | shock transcription factor, other eukaryote; heat stress transcription factor B-2c, |
| | | heat shock transcription factor, other eukaryote; uncharacterized |
| | | LOC100280131, HSP20 family protein; 16.0 kDa heat shock protein, |
| | | peroxisomal, HSP20 family protein; 16.9 kDa class I heat shock protein 1, |
| | | HSP20 family protein; 17.9 kDa class I heat shock protein, HSP20 family |
| | | protein; 22.0 kDa class IV heat shock protein, HSP20 family protein; class I |
| | | heat shock protein 3, HSP20 family protein; class II heat shock protein, HSP20 |
| | | family protein; hsp18a; 17.0 kDa class II heat shock protein, HSP20 family |
| | | protein; hsp18f; 17.5 kDa class II heat shock protein, HSP20 family protein; |
| | | hsp22; low molecular weight heat shock protein precursor, HSP20 family |
| | | protein; TIDP2749; uncharacterized protein LOC100283766, HSP20 family |
| | | protein; uncharacterized protein LOC100281798, HSP20 family protein; |
| | | uncharacterized protein LOC100383105, hsp70-interacting protein; Fes1A, |
| | | hsp70-interacting protein; uncharacterized protein LOC100275241, KDEL- |
| | | tailed cysteine endopeptidase [EC: 3.4.22.-]; hypothetical protein, large proline- |
| | | rich protein BAG6; uncharacterized protein LOC110600224 isoform X1, |
| | | molecular chaperone DnaJ; DNAJ heat shock family protein, molecular |
| | | chaperone HtpG; hsp82; heat shock protein 82, molecular chaperone HtpG; |
| | | HSP90-2; HSP protein, molecular chaperone HtpG; pco150589a; |
| | | uncharacterized protein LOC100272368, molecular chaperone HtpG; |
| | | pco153543(105); uncharacterized protein LOC100384473, |
| | | oligosaccharyltransferase complex subunit alpha (ribophorin I); dolichyl- |
| | | diphosphooligosaccharideprotein glycosyltransferase 67 kDasubunit precursor, |
| | | oligosaccharyltransferase complex subunit beta; dolichyl- |
| | | diphosphooligosaccharideprotein glycosyltransferase 48 kDasubunit precursor, |
| | | peptidyl-prolyl cis-trans isomerase-like 3 [EC: 5.2.1.8]; TIDP9202; |
| | | uncharacterized protein LOC100280578, phytepsin [EC: 3.4.23.40]; aspartic |
| | | proteinase oryzasin-1, pleiotropic regulator 1; uncharacterized protein |
| | | LOC9311097, protease IV [EC: 3.4.21.-]; putative signal peptide peptidase |
| | | family protein, protein disulfide-isomerase A1 [EC: 5.3.4.1]; PDIL1-1; protein |
| | | disulfide-isomerase precursor, protein disulfide-isomerase A6 [EC: 5.3.4.1]; |
| | | pdi8; protein disulfide isomerase 8 precursor, protein transport protein SEC61 |
| | | subunit beta; protein transport protein Sec61 subunit beta, RING-H2 zinc finger |
| | | protein RHA1; E3 ubiquitin-protein ligase RHA1B, serine/threonine-protein |
| | | kinase PBS1 [EC: 2.7.11.1]; serine/threonine-protein kinase PBS1-like, stress- |
| | | induced-phosphoprotein 1; Hsp70-Hsp90 organizing protein 3, stress-induced- |
| | | phosphoprotein 1; hsp70-Hsp90 organizing protein, stress-induced- |
| | | phosphoprotein 1; pco123267; uncharacterized protein LOC100285569, |
| | | thioredoxin 1; uncharacterized protein LOC606487, transcription factor TGA; |
| | | transcription factor TGA7, putative, ubiquitin-conjugating enzyme E2 D |
| | | [EC: 2.3.2.23]; ubiquitin-conjugating enzyme E2 28. |
| Heat Shock | Formulation 1 | ATP-dependent Clp protease ATP-binding subunit ClpB; hsp101; heat-shock |
| Proteins and | | protein 101, ATP-dependent Clp protease ATP-binding subunit ClpB; |
| Transcription | | uncharacterized protein LOC100217072 isoform X1, calreticulin; calreticulin- |
| Factors | | like, calreticulin; gpm494; uncharacterized protein LOC100382232 precursor, |
| | | DnaJ homolog subfamily A member 2; putative dnaJ chaperone family protein, |
| | | DnaJ homolog subfamily A member 5; predicted protein, DnaJ homolog |
| | | subfamily B member 11; dnaJ homolog subfamily B member 11-like precursor, |
| | | DnaJ homolog subfamily B member 4; dnaJ homolog subfamily B member 4, |
| | | DnaJ homolog subfamily B member 4; dnaJ protein, DnaJ homolog subfamily |
| | | B member 4; uncharacterized protein LOC100382308, E3 ubiquitin-protein |
| | | ligase ATL10/75/76/77/78 [EC: 2.3.2.27]; RING-H2 finger protein ATL74, E3 |
| | | ubiquitin-protein ligase ATL10/75/76/77/78 [EC: 2.3.2.27]; RING-H2 finger |
| | | protein ATL78-like, E3 ubiquitin-protein ligase ATL41 [EC: 2.3.2.27]; probable |
| | | E3 ubiquitin-protein ligase ATL44, E3 ubiquitin-protein ligase RNF115/126 |
| | | [EC: 2.3.2.27]; E3 ubiquitin-protein ligase RING1-like, FK506-binding protein |
| | | 4/5 [EC: 5.2.1.8]; uncharacterized protein LOC100283417, glutathione S- |
| | | transferase [EC: 2.5.1.18]; csu454(gst); uncharacterized protein LOC100282447, |
| | | glutathione S-transferase [EC: 2.5.1.18]; GST39; glutathione transferase39, |
| | | glutathione S-transferase [EC: 2.5.1.18]; IN2-1 protein, heat shock 70 kDa |
| | | protein 1/2/6/8; heat shock 70 kDa protein-like, heat shock 70 kDa protein |
| | | 1/2/6/8; heat shock cognate 70 kDa protein, heat shock 70 kDa protein 1/2/6/8; |
| | | heat shock protein 1, heat shock 70 kDa protein 1/2/6/8; pco083553; heat shock |
| | | cognate 70 kDa protein 2, heat shock 70 kDa protein 1/2/6/8; probable mediator |
| | | of RNA polymerase II transcription subunit 37c, heat shock 70 kDa protein 4; |
| | | heat shock 70 kDa protein 14, heat shock 70 kDa protein 4; heat shock 70 kDa |
| | | protein 15, heat shock 70 kDa protein 4; heat shock 70 kDa protein 4, heat shock |
| | | protein 90 kDa beta; endoplasmin precursor, heat shock protein 90 kDa beta; |
| | | shpl1; shepherd-like 1 precursor, heat shock transcription factor, other |
| | | eukaryote; heat shock factor protein 4, heat shock transcription factor, other |
| | | eukaryote; heat shock factor protein 7, heat shock transcription factor, other |
| | | eukaryote; heat shock factor protein HSF30, heat shock transcription factor, |
| | | other eukaryote; heat stress transcription factor B-2c, heat shock transcription |
| | | factor, other eukaryote; uncharacterized LOC100280131, HSP20 family |
| | | protein; 16.9 kDa class I heat shock protein 1, HSP20 family protein; 17.5 kDa |
| | | class I heat shock protein, HSP20 family protein; 17.9 kDa class I heat shock |
| | | protein, HSP20 family protein; class I heat shock protein 3, HSP20 family |
| | | protein; class II heat shock protein, HSP20 family protein; hsp18a; 17.0 kDa |
| | | class II heat shock protein, HSP20 family protein; hsp18c; 17.8 kDa class II |
| | | heat shock protein, HSP20 family protein; hsp22; low molecular weight heat |
| | | shock protein precursor, HSP20 family protein; uncharacterized protein |
| | | LOC100281798, HSP20 family protein; uncharacterized protein |
| | | LOC100383105, hsp70-interacting protein; uncharacterized protein |
| | | LOC100275241, maleylacetoacetate isomerase [EC: 5.2.1.2]; gst18; glutathione |
| | | S-transferase GST 18, molecular chaperone HtpG; hsp82; heat shock protein 82, |
| | | molecular chaperone HtpG; HSP90-2; HSP protein, molecular chaperone HtpG; |
| | | pco153543(105); uncharacterized protein LOC100384473, phytepsin |
| | | [EC: 3.4.23.40]; aspartic proteinase oryzasin-1, phytepsin [EC: 3.4.23.40]; |
| | | aspartic proteinase oryzasin-1, protein disulfide-isomerase A6 [EC: 5.3.4.1]; |
| | | pdi8; protein disulfide isomerase 8 precursor, serine/threonine-protein kinase |
| | | PBS1 [EC: 2.7.11.1]; serine/threonine-protein kinase PBS1-like, stress-induced- |
| | | phosphoprotein 1; hsp70-Hsp90 organizing protein, transcription factor TGA; |
| | | transcription factor TGA7, putative, ubiquitin-conjugating enzyme E2 O |
| | | [EC: 2.3.2.24]; probable ubiquitin-conjugating enzyme E2 23, xylem cysteine |
| | | proteinase [EC: 3.4.22.-]; hypothetical protein. |
| Heat Shock | Formulation 3 | HSP70 family proteins; HSP90 (endoplasmin/HSP90-2); ATP-dependent Clp |
| Proteins and | | protease ClpB (HSP101); DnaJ homolog subfamily A and B proteins; |
| Transcription | | chaperonin GroEL/GroES |
| Factors | | |
| Aquaporin | Urea | aquaporin TIP; tip4a; aquaporin TIP4-1. |
| Biosynthesis | | |
| Aquaporin | Formulation 2 | aquaporin PIP; PIP1-4; aquaporin PIP1-3/PIP1-4, aquaporin TIP; tip1; |
| Biosynthesis | | aquaporin TIP1-1, aquaporin PIP; pip1b; aquaporin PIP1-2, aquaporin TIP; |
| | | tip2a; aquaporin TIP2-1, aquaporin PIP; pip2c; aquaporin PIP2-3, aquaporin |
| | | NIP; nip2b; aquaporin NIP2-2, aquaporin PIP; probable aquaporin PIP2-7. |
| Aquaporin | Formulation 1 | aquaporin PIP; PIP1-4; aquaporin PIP1-3/PIP1-4, aquaporin PIP; pip2e; |
| Biosynthesis | | aquaporin PIP2-1, aquaporin PIP; pip2f; aquaporin PIP2-6, aquaporin TIP; tip1; |
| | | aquaporin TIP1-1, aquaporin PIP; pip2c; aquaporin PIP2-3. |
| Aquaporin | Formulation 3 | tip1, aquaporin TIP1-1; tip2b, aquaporin TIP2-2; nip3a, aquaporin NIP3-1; |
| Biosynthesis | | tip4d, aquaporin TIP4-4; pip1-4, aquaporin PIP1-3/PIP1-4; pip1b, aquaporin |
| | | PIP1-2; pip2c, aquaporin PIP2-3; pip2-7, aquaporin PIP2-7 |
| Chromatin - | Formulation 2 | DNA gyrase subunit A [EC: 5.6.2.2]; probable DNA gyrase subunit A, |
| Epigenetics | | chloroplastic/mitochondrial, lysine-specific demethylase 8 |
| | | [EC: 1.14.11.27]; lysine-specific demethylase JMJ30 isoform X1. |
| Chromatin - | Formulation 1 | lysine-specific demethylase 8 [EC: 1.14.11.27]; lysine-specific demethylase |
| Epigenetics | | JMJ30 isoform X1, [histone H3]-lysine4/36 N-trimethyltransferase SMYD |
| | | [EC: 2.1.1.354 2.1.1.357]; histone-lysine N-methyltransferase ASHR1, |
| | | coronatine-insensitive protein 1; uncharacterized protein LOC100382859. |
| Chromatin - | Formulation 3 | lysine-specific demethylase JMJ30 |
| Epigenetics | | |
| C4 | Formulation 2 | malate dehydrogenase [EC: 1.1.1.37]; malate dehydrogenase, cytoplasmic-like, |
| Photosynthesis | | fumarate hydratase, class II [EC: 4.2.1.2]; pco106335; uncharacterized protein |
| and | | LOC100279871. |
| Metabolism | | |
| C4 | Formulation 1 | malate dehydrogenase [EC: 1.1.1.37]; malate dehydrogenase, cytoplasmic-like. |
| Photosynthesis | | |
| and | | |
| Metabolism | | |
| C4 | Formulation 3 | ribulose-bisphosphate carboxylase large chain; phosphoenolpyruvate |
| Photosynthesis | | carboxylase; malate dehydrogenase (cytoplasmic-like); fumarate hydratase |
| and | | (class II) |
| Metabolism | | |
| Trehalose | Urea | trehalose 6-phosphate synthase/phosphatase [EC: 2.4.1.15 3.1.3.12]; |
| | | alpha,alpha-trehalose-phosphate synthase [UDP-forming] 6, trehalose 6- |
| | | phosphate synthase/phosphatase [EC: 2.4.1.15 3.1.3.12]; uncharacterized protein |
| | | LOC103654185, trehalose 6-phosphate synthase/phosphatase [EC: 2.4.1.15 |
| | | 3.1.3.12]; gpm600; uncharacterized protein LOC100281927, inositol 3-alpha- |
| | | galactosyltransferase [EC: 2.4.1.123]; gols2; galactinol synthase 2, inositol 3- |
| | | alpha-galactosyltransferase [EC: 2.4.1.123]; galactinol synthase 1. |
| Trehalose | Formulation 2 | trehalose 6-phosphate synthase/phosphatase [EC: 2.4.1.15 3.1.3.12]; trehalose-6- |
| | | phosphate synthase, trehalose 6-phosphate synthase/phosphatase [EC: 2.4.1.15 |
| | | 3.1.3.12]; alpha,alpha-trehalose-phosphate synthase [UDP-forming] 1, trehalose |
| | | 6-phosphate synthase/phosphatase [EC: 2.4.1.15 3.1.3.12]; probable alpha,alpha- |
| | | trehalose-phosphate synthase [UDP-forming] 9, trehalose 6-phosphate |
| | | synthase/phosphatase [EC: 2.4.1.15 3.1.3.12]; probable alpha,alpha-trehalose- |
| | | phosphate synthase [UDP-forming] 10, trehalose 6-phosphate |
| | | synthase/phosphatase [EC: 2.4.1.15 3.1.3.12]; uncharacterized protein |
| | | LOC103654185. |
| Trehalose | Formulation 3 | trehalose-6-phosphate synthase/phosphatase; alpha,alpha-trehalose-phosphate |
| | | synthase [UDP-forming] (TPS1, TPS9, TPS10) |

### Example 3: Improvement in Abiotic and Biotic Stress Physiology

Studies were conducted to determine whether treatment of plants with a composition described herein improves abiotic and biotic stress physiology, in addition to the molecular, biochemical, and physiological mechanisms associated with proteostasis and preservation of protein function. Corn plants (Zea mays, V6 growth stage) were grown under controlled greenhouse conditions and treated foliarly with the formulation 2. Root and leaf tissues were harvested at approximately 4 hours, 24 hours, and 96 hours after treatment (HAT) and subjected to transcriptomic analysis using full-length RNA sequencing. Differentially expressed genes were identified using thresholds of log 2 fold change≥1.0 and false discovery rate (FDR)≤0.05, with relaxed thresholds applied for pathway-level enrichment.

At 4 HAT, root tissue exhibited strong modulation of stress-associated signaling pathways, including activation of MAPK signaling components and WRKY transcription factors, consistent with early immune and stress perception. Notably, genes encoding heat shock proteins and chaperones, including multiple members of the HSP20 and HSP70 families, were downregulated rather than upregulated at this early timepoint, with log 2 fold changes ranging from approximately −1.0 to −2.4. This reduction in chaperone demand occurred concurrently with activation of defense signaling pathways and redox-related genes, indicating that formulation 2 induces a stress-primed physiological state that limits proteotoxic stress rather than inducing damage-driven protein denaturation.

This molecular profile was directly corroborated by biochemical measurements from the metabolic studies. Leaves of plants treated with formulation 2 showed statistically significant increases in antioxidant enzyme activity, including superoxide dismutase (SOD), catalase (CAT), and ascorbate peroxidase (APX), compared to urea-only controls. Increased activity of these enzymes demonstrates enhanced detoxification of reactive oxygen species, which are primary causes of protein oxidation and loss of enzymatic functionality during abiotic and biotic stress. The combined transcriptomic and enzymatic data therefore demonstrate preservation of protein function through reduced oxidative burden rather than increased chaperone demand.

At 24 HAT, transcriptomic data demonstrated continued reduction or normalization of heat shock protein expression in both root and leaf tissues, while genes associated with protein processing in the endoplasmic reticulum, ribosomal function, and translational capacity were significantly upregulated, particularly in leaf tissue. Ribosomal proteins and ER-associated folding machinery showed log 2 fold changes of approximately +1.2 to +1.5, indicating enhanced protein synthesis and quality control capacity under non-stress conditions. At 96 HAT, stress-associated gene expression had largely resolved, while photosynthesis-associated genes and growth pathways were restored. Physiologically, this resolution was reflected in sustained photosystem performance, with chlorophyll a fluorescence measurements (Fv/Fm, PIabs, PItotal) remaining high over the 1-28 DAA observation window.

Collectively, these transcriptomic, biochemical, and physiological datasets demonstrate that the formulation 2 composition improves abiotic and biotic stress physiology by inducing transient stress signaling, enhancing antioxidant protection, and preserving protein functionality without imposing a sustained growth penalty.

### Example 4: Improved Translocation of Sugars, Nutrients, and Water Through Upregulation of Aquaporins and Trehalose-6-Phosphate

Studies were conducted to evaluate whether treatment with formulation 2 enhances internal translocation of sugars, nutrients, and water in addition to regulation of transporter networks and carbon signaling pathways. Corn plants were grown as described in Example 3, after which root and shoot tissues were collected at 4, 24, and 96 HAT for transcriptomic analysis, and physiological measurements were conducted from 1 to 28 days after application (DAA).

At 4 HAT, root tissue exhibited significant upregulation of aquaporin genes across multiple families, including plasma membrane intrinsic proteins (PIPs) and tonoplast intrinsic proteins (TIPs), with log 2 fold changes ranging from approximately +1.0 to +1.55. Nodulin-26-like intrinsic proteins (NIPs), associated with transport of small neutral solutes such as urea and boron, were also differentially expressed. In parallel, ABC transporter families involved in intracellular trafficking and long-distance transport showed increased expression, supporting redistribution of assimilated resources.

Transcriptomic enrichment analysis further identified modulation of carbohydrate sensing and signaling pathways, including differential expression of genes associated with trehalose-6-phosphate metabolism. Because trehalose-6-phosphate is a central regulator of carbon allocation and source-sink balance, this molecular response indicates enhanced coordination between sugar production and transport.

Physiological measurements provided functional validation of these molecular changes.

Chlorophyll a fluorescence analyses showed sustained increases in photosynthetic performance indexes (PIabs and PItotal), increased electron transport per reaction center (ETo/RC), and reduced energy dissipation (DIo/RC) in formulation 2-treated plants relative to urea controls across the 1-28 DAA period. Total pigment measurements demonstrated delayed chlorophyll loss, with chlorophyll concentrations remaining approximately 1.03 mg g⁻¹ fresh matter at 28 DAA for urea+formulation 2 plants versus approximately 0.43 mg g⁻¹ in untreated controls. Sustained pigment content and enhanced photosynthetic efficiency provide increased sugar supply for phloem translocation.

By 24 HAT, aquaporin expression remained elevated in root tissue and showed systemic propagation to shoot tissues, with increased expression of sugar, amino acid, and mineral nutrient transporters in leaves. By 96 HAT, transporter expression partially normalized but remained elevated relative to controls, indicating establishment of a sustained transport-optimized physiological state. Together, these data demonstrate that formulation 2 improves translocation of water, sugars, and nutrients through coordinated upregulation of aquaporins, carbohydrate signaling pathways, and sustained photosynthetic performance.

### Example 5: Improved Nutrient Use Efficiency Through Upregulation of Amino Acid Biosynthesis

Studies were conducted to determine whether formulation 2 improves nutrient use efficiency through enhanced nitrogen assimilation and amino acid biosynthesis rather than increased nutrient input. Corn plants were grown as described in Example 3 under equivalent nitrogen availability, followed by transcriptomic, enzymatic, and growth analyses.

At 4 HAT, root tissue exhibited strong upregulation of nitrogen assimilation and amino acid biosynthesis pathways. Genes encoding glutamine synthetase (GS), glutamate synthase (GOGAT), nitrate reductase (NR), and nitrite reductase showed log 2 fold changes ranging from approximately +1.0 to +1.8. Asparagine synthase and branched-chain amino acid biosynthesis genes were also upregulated, indicating increased conversion of inorganic nitrogen into organic nitrogen forms.

These transcriptional changes were directly validated by biochemical assays. Leaf nitrate reductase activity was significantly higher in formulation 2-treated plants compared to urea alone, demonstrating increased nitrate reduction capacity. Glutamine synthetase activity was likewise significantly increased, confirming enhanced assimilation of ammonium into glutamine, the central donor of nitrogen to amino acid biosynthesis.

These biochemical improvements translated into increased plant growth under equivalent nutrient input. At 28 DAA, shoot dry mass increased from approximately 19.50 g in urea-only plants to approximately 21.11 g in urea+formulation 2 plants, and root dry mass increased from 10.28 g to 13.16 g. Plant height increased from 53.09 cm to 55.96 cm at 28 DAA. Because applied nitrogen was matched across treatments, the increased biomass and height demonstrate improved nutrient use efficiency driven by enhanced nitrogen assimilation rather than increased fertilizer availability.

### Example 6: Improved Nutrient Use Efficiency and Fertilizer Performance in DAP Technologies

Studies were conducted to evaluate whether the formulation 3 enhances the physiological and molecular utilization of diammonium phosphate (DAP) fertilizers. Corn plants were grown under controlled greenhouse conditions with DAP supplied as the primary nitrogen and phosphorus source. Treatments included DAP alone and DAP combined with foliar application of the formulation 3 composition at the recommended rate.

At 4 HAT, transcriptomic analysis showed significant upregulation of nutrient transporter genes in root tissue of plants treated with formulation 3 compared to DAP alone. Phosphate transporters, nitrate and ammonium transporters, aquaporins, and ABC transporters showed log 2 fold changes ranging from approximately +0.8 to +1.5, indicating enhanced uptake and early redistribution of DAP-derived nutrients.

At 24 HAT, transcriptomic enrichment revealed increased expression of photosynthesis-related genes, plastid-associated energy metabolism pathways, and amino acid biosynthesis genes.

Carbon metabolism and ribosomal protein synthesis pathways were also enriched, indicating improved integration of nutrient availability with metabolic demand. By 96 HAT, transporter and metabolic gene expression stabilized at levels supporting sustained nutrient assimilation.

These molecular responses are consistent with the metabolic datasets showing enhanced nitrogen assimilation, improved photosynthetic performance, and increased biomass accumulation under treatments with formulation 2. Collectively, these data demonstrate that formulation 3 enhances fertilizer performance by improving uptake, translocation, and assimilation of applied nitrogen and phosphorus rather than increasing fertilizer application rates.

While the foregoing written description of the invention enables one of ordinary skill to make and use what is considered presently to be the best mode thereof, those of ordinary skill will understand and appreciate the existence of variations, combinations, and equivalents of the specific embodiment, method, and examples herein. The invention should therefore not be limited by the above-described embodiment, method, and examples, but by all embodiments and methods within the scope and spirit of the invention.

1-33. (canceled)

34. An agrochemical composition for inducing differential gene expression in a plant, the agrochemical composition comprising: (a) at least one plant extract, (b) at least one weak acid, (c) at least one oligosaccharide, (d) at least one amino acid or amino acid source, and (e) one or more adjuvants;
wherein the composition upregulates expression of at least one gene associated with a biochemical or physiological pathway selected from the group consisting of amino acid transport and metabolism, antioxidant activity, aquaporin biosynthesis, auxin biosynthesis and signaling, C4 photosynthesis and metabolism, carotenoid biosynthesis, chromatin epigenetics, heat shock proteins, transcription factors, phenylpropanoid biosynthesis, photosynthesis-related pathways, transporters, trehalose metabolism, WRKY transcription factors, and a combination thereof.

35. The composition of claim 34, further comprising a nutrient particle or a plurality of nutrient particles, wherein the nutrient particle comprises a plant nutrient selected from the group consisting of nitrogen, phosphorus, potassium, compounds thereof, salts thereof, and a combination thereof.

36. The composition of claim 35, wherein the nutrient particle comprises a nitrogen-containing compound selected from the group consisting of urea, ammonium salts, nitrate salts, and combinations thereof.

37. The composition of claim 35, wherein the nutrient particle comprises a phosphorus-containing compound selected from the group consisting of phosphates, polyphosphates, and combinations thereof.

38. The composition of claim 35, wherein the weak acid is selected from the group consisting of citric acid, malic acid, lactic acid, formic acid, acetic acid, benzoic acid, carbonic acid, and combinations thereof.

39. The composition of claim 35, wherein the oligosaccharide is a chito oligosaccharide.

40. The composition of claim 35, wherein the amino acid or amino acid source is an extract comprising an amino acid.

41. A method for increasing nutrient use efficiency and internal nutrient translocation in a plant, comprising applying an agrochemical composition comprising: (a) at least one plant extract, (b) at least one weak acid, (c) at least one oligosaccharide, (d) at least one amino acid or amino acid source, and (e) one or more adjuvants,
wherein the composition induces differential gene expression of a pathway associated with a pathways selected from the group consisting of nutrient assimilation, membrane permeability, transport proteins, and water movement, thereby enhancing uptake, assimilation, and redistribution of nutrients within the plant.

42. The method of claim 41, wherein the composition further comprises a nutrient particle selected from the group consisting of nitrogen, phosphorus, potassium, compounds thereof, salts thereof, and a combination thereof.

43. The method of claim 41, wherein differential gene expression comprises increased expression of enzymes selected from the group consisting of nitrate reductase, nitrite reductase, glutamine synthetase, glutamate synthase, and a combination thereof.

44. The method of claim 41, wherein nutrient use efficiency is increased without an increase in external nutrient application rates.

45. The method of claim 41, wherein differential gene expression comprises increased expression of aquaporins of genes selected from the group consisting of plasma membrane intrinsic proteins (PIPs), tonoplast intrinsic proteins (TIPs), nodulin-26-like intrinsic proteins (NIPs), and a combination thereof.

46. The method of claim 41, wherein differential gene expression comprises increased expression of transport proteins selected from the group consisting of nitrate transporters, phosphate transporters, potassium transporters, ATP-binding cassette transporters, and a combination thereof.

47. The method of claim 41, wherein enhanced translocation comprises long-distance movement of water, minerals, or photo assimilates from source tissues to sink tissues.

48. A method for increasing abiotic and biotic stress resilience in a plant without a sustained growth penalty, comprising applying an agrochemical composition comprising: (a) at least one plant extract, (b) at least one weak acid, (c) at least one oligosaccharide, (d) at least one amino acid or amino acid source, and (e) one or more adjuvants,
wherein the composition induces transient differential gene expression of immune, antioxidant, proteostasis, and stress-associated regulatory pathways while maintaining expression of growth-associated metabolic and photosynthesis-related genes.

49. The method of claim 48, wherein the stress is selected from the group consisting of drought stress, heat stress, cold stress, salinity stress, nutrient deficiency stress, oxidative stress, biotic stress, and combinations thereof.

50. The method of claim 48, wherein differential gene expression comprises increased expression of antioxidant enzymes selected from the group consisting of superoxide dismutase, catalase, ascorbate peroxidase, glutathione-associated enzymes, and combinations thereof.

51. The method of claim 48, wherein immune-associated signaling includes WRKY transcription factor-mediated signaling.

52. The method of claim 48, wherein stress-associated signaling pathways are transiently activated within about 4 to about 24 hours following application.

53. The method of claim 48, wherein application results in increased biomass accumulation, enhanced reproductive development, increased yield, or combinations thereof under equivalent nutrient availability.
